SEQUENCE LISTING (59 sequences)

SEQ ID NO: 1 - protein, 182 aa, Homo sapiens
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 2 - protein, 182 aa, Mus musculus
  MISC_FEATURE: What is shown is the portion of the Mus musculus NK1 protein thatis homogolous with the homo sapien NK1
YAEGQKKRRNTLHEFKKSAKTTLTKEDPLLKIKTKKVNSADECANRCIRNRGFTFTCKAFVFDKSRKRCYWYPFNSMSSGVKKGFGHEFDLYENKDYIRNCIIGKGGSYKGTVSITKSGIKCQPWNSMIPHEHSFLPSSYRGKDLQENYCRNPRGEEGGPWCFTSNPEVRYEVCDIPQCSEV

SEQ ID NO: 3 - protein, 182 aa, Rattus norvegicus
  MISC_FEATURE: What is shown is the portion of the Rattus norvegicus NK1 protein that is homogolous with the homo sapien NK1
YAEGHKKRRNTIHEFKKSAKTTLIKIDPALKIKTKKVNTADQCANRCTRNNGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 4 - protein, 182 aa, Bos taurus
  MISC_FEATURE: What is shown is the portion of the Bos taurus NK1 protein that is homogolous with the homo sapien NK1
YAEGQKKRRNTLHEFKRSAKTTLIKEDPLLKIKTKKMNTADQCANRCIRNKGLPFTCKAFVFDKARKRCLWFPFNSMSSGVKKEFGHEFDLYENKDYIRNCIIGKGGSYKGTVSITKSGIKCQPWNSMIPHEHSFLPSSYRGKDLQENYCRNPRGEEGGPWCFTSNPEVRYEVCDIPQCSEV

SEQ ID NO: 5 - protein, 182 aa, Felis catus
  MISC_FEATURE: What is shown is the portion of the Felis catus NK1 protein that is homogolous with the homo sapien NK1
YAEGQKKRRNTLHEFKKSAKTTLIKEDPLLKIKTKKMNTADQCANRCIRNKGLPFTCKAFVFDKARKRCLWFPFNSMTSGVKKEFGHEFDLYENKDYIRNCIIGKGGSYKGTVSITKSGIKCQPWNSMIPHEHSFLPSSYRGKDLQENYCRNPRGEEGGPWCFTSNPEVRYEVCDIPQCSEV

SEQ ID NO: 6 - protein, 182 aa, Canis familiaris
  MISC_FEATURE: What is shown is the portion of the Canis familiaris NK1 protein that is homogolous with the homo sapien NK1
RAEGQKKRRNTLHEFKKSAKTTLIKEDPLLKIKTKKMNTADQCANRCIRNKGLPFTCKAFVFDKARKRCLWFPFNSMTSGVKKEFGHEFDLYENKDYIRNCIIGKGGSYKGTVSITKSGIKCQPWNSMIPHEHSFLPSSYRGKDLQENYCRNPRGEEGGPWCFTSNPEVRYEVCDIPQCSEV

SEQ ID NO: 7 - protein, 182 aa, Gallus gallus
  MISC_FEATURE: What is shown is the portion of the Gallus gallus NK1 protein that is homogolous with the homo sapien NK1
AAEGKGKRRNPLHDYKKTGELMLIKVNKTLEVKTKLLNTTEQCAKRCSRNKGLSFTCKAFAYDRVTKRCHWLSFNSLTNGVRKKQDHAFDLFEKKDYVRNCIIGKGAEYKGTISITKSGIQCQAWNSMIPHEHSFLPSSYRGKDLRENYCRNPRGEEGGPWCFTTSPQMRHEVCDIPLCSEV

SEQ ID NO: 8 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDAIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 9 - protein, 182 aa, Artificial Sequence
  Human mutated peptide
YAEGQRKRRNTIHEFKKSAKTTLIKIDPALKIKTKKVNTADQCANRCTRNKGLPFTCAAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 10 - protein, 182 aa, Artificial Sequence
  Human mutated peptide
YAEGQRKRRNTIHEFKKSAKTTLIKIDPALKIKTKKVNTADQCANRCTRNKGLPFTCAAFVFDKARKQCLWFPFNSMSSGVKKEFGHEFDLYENKAYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 11 - protein, 182 aa, Artificial Sequence
  Human mutated peptide
YAEGQRKRRNTIHEFKKSAKTTLIKIDPALKIKTKKVNTADQCANRCTRNKGLPFTCAAFVFDKARKQCLWFPFNSMSSGVKKEFGHEFDLYENKDYIRACIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 12 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKAYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 13 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRACIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 14 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDXIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 15 - protein, 182 aa, Artificial Sequence
  Human mutated peptide
YAEGQRKRRNTIHEFKKSAKTTLIKIDPALKIKTKKVNTADQCANRCTRNKGLPFTCXAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 16 - protein, 182 aa, Artificial Sequence
  Human mutated peptide
YAEGQRKRRNTIHEFKKSAKTTLIKIDPALKIKTKKVNTADQCANRCTRNKGLPFTCXAFVFDKARKQCLWFPFNSMSSGVKKEFGHEFDLYENKXYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 17 - protein, 182 aa, Artificial Sequence
  Human mutated peptide
YAEGQRKRRNTIHEFKKSAKTTLIKIDPALKIKTKKVNTADQCANRCTRNKGLPFTCXAFVFDKARKQCLWFPFNSMSSGVKKEFGHEFDLYENKDYIRXCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 18 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKXYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 19 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRXCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 20 - protein, 182 aa, Artificial Sequence
  Human mutated peptide
YAEGQRKRRNTIHEFKKSAKTTLIKIDPALKIKTKKVNTADQCANRCTRAKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 21 - protein, 182 aa, Artificial Sequence
  Human mutated peptide
YAEGQRKRRNTIHEFKKSAKTTLIKIDPALKIKTKKVNTADQCANRCTRNKGLPAACAAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 22 - protein, 182 aa, Artificial Sequence
  Human mutated peptide
YAEGQRKRRNTIHEFKKSAKTTLIKIDPALKIKTKKVNTADQCANRCTRAKGLPAACAAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 23 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENADYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 24 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIANCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 25 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKAAIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 26 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKAYIRACIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 27 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKAAIRACIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 28 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTASATKSGIKCQPWSSMIPHEHSFLPSSYRGKDLQENYCRNPRGEEGGPWCFTSNPEVRYEVCDIPQCSEV

SEQ ID NO: 29 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGAVAATKSGIKCQPWSSMIPHEHSFLPSSYRGKDLQENYCRNPRGEEGGPWCFTSNPEVRYEVCDIPQCSEV

SEQ ID NO: 30 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYAGAVAATKSGIKCQPWSSMIPHEHSFLPSSYRGKDLQENYCRNPRGEEGGPWCFTSNPEVRYEVCDIPQCSEV

SEQ ID NO: 31 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYAGAASATKSGIKCQPWSSMIPHEHSFLPSSYRGKDLQENYCRNPRGEEGGPWCFTSNPEVRYEVCDIPQCSEV

SEQ ID NO: 32 - protein, 182 aa, Artificial Sequence
  Human mutated peptide
YAEGQRKRRNTIHEFKKSAKTTLIKIDPALKIKTKKVNTADQCANRCTRNKGLPAACAAFVFDKARKQCLWFPFNSMSSGVKKEFGHEFDLYENKAYIRACIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 33 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKAYIRNCIIGKGRSYAGTVSITKSGIKCQPWSSMIPHEHSFLPSSYRGKDLQENYCRNPRGEEGGPWCFTSNPEVRYEVCDIPQCSEV

SEQ ID NO: 34 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKAYIRNCIIGKGRSYAGAASATKSGIKCQPWSSMIPHEHSFLPSSYRGKDLQENYCRNPRGEEGGPWCFTSNPEVRYEVCDIPQCSEV

SEQ ID NO: 35 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKAYIRNCIIGKGRSYAGAVAATKSGIKCQPWSSMIPHEHSFLPSSYRGKDLQENYCRNPRGEEGGPWCFTSNPEVRYEVCDIPQCSEV

SEQ ID NO: 36 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKAYIRACIIGKGRSYAGAASATKSGIKCQPWSSMIPHEHSFLPSSYRGKDLQENYCRNPRGEEGGPWCFTSNPEVRYEVCDIPQCSEV

SEQ ID NO: 37 - protein, 182 aa, Artificial Sequence
  Human mutated peptide
YAEGQRKRRNTIHEFKKSAKTTLIKIDPALKIKTKKVNTADQCANRCTRXKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 38 - protein, 182 aa, Artificial Sequence
  Human mutated peptide
YAEGQRKRRNTIHEFKKSAKTTLIKIDPALKIKTKKVNTADQCANRCTRNKGLPXXCXAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 39 - protein, 182 aa, Artificial Sequence
  Human mutated peptide
YAEGQRKRRNTIHEFKKSAKTTLIKIDPALKIKTKKVNTADQCANRCTRXKGLPXXCXAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 40 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENXDYIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 41 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIXNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 42 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKXXIRNCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 43 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKXYIRXCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 44 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKXXIRXCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 45 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTXSXTKSGIKCQPWSSMIPHEHSFLPSSYRGKDLQENYCRNPRGEEGGPWCFTSNPEVRYEVCDIPQCSEV

SEQ ID NO: 46 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGXVXXTKSGIKCQPWSSMIPHEHSFLPSSYRGKDLQENYCRNPRGEEGGPWCFTSNPEVRYEVCDIPQCSEV

SEQ ID NO: 47 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYXGXVXXTKSGIKCQPWSSMIPHEHSFLPSSYRGKDLQENYCRNPRGEEGGPWCFTSNPEVRYEVCDIPQCSEV

SEQ ID NO: 48 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYXGXXSXTKSGIKCQPWSSMIPHEHSFLPSSYRGKDLQENYCRNPRGEEGGPWCFTSNPEVRYEVCDIPQCSEV

SEQ ID NO: 49 - protein, 182 aa, Artificial Sequence
  Human mutated peptide
YAEGQRKRRNTIHEFKKSAKTTLIKIDPALKIKTKKVNTADQCANRCTRNKGLPXXCXAFVFDKARKQCLWFPFNSMSSGVKKEFGHEFDLYENKXYIRXCIIGKGRSYKGTVSITKSGIKCQPWSSMIPHEHSFLPSSYRGKDLQENYCRNPRGEEGGPWCFTSNPEVRYEVCDIPQCSEV

SEQ ID NO: 50 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKXYIRNCIIGKGRSYXGTVSITKSGIKCQPWSSMIPHEHSFLPSSYRGKDLQENYCRNPRGEEGGPWCFTSNPEVRYEVCDIPQCSEV

SEQ ID NO: 51 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKXYIRNCIIGKGRSYXGXXSXTKSGIKCQPWSSMIPHEHSFLPSSYRGKDLQENYCRNPRGEEGGPWCFTSNPEVRYEVCDIPQCSEV

SEQ ID NO: 52 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKXYIRNCIIGKGRSYXGXVXXTKSGIKCQPWSSMIPHEHSFLPSSYRGKDLQENYCRNPRGEEGGPWCFTSNPEVRYEVCDIPQCSEV

SEQ ID NO: 53 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKXYIRXCIIGKGRSYXGXXSXTKSGIKCQPWSSMIPHEHSFLPSSYRGKDLQENYCRNPRGEEGGPWCFTSNPEVRYEVCDIPQCSEV

SEQ ID NO: 54 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTRSITKSGIKCQPWSSMIPHEHSFLPSSYRGKDLQENYCRNPRGEEGGPWCFTSNPEVRYEVCDIPQCSEV

SEQ ID NO: 55 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTXSITKSGIKCQPWSSMIPHEHSFLPSSYRGKDLQENYCRNPRGEEGGPWCFTSNPEVRYEVCDIPQCSEV

SEQ ID NO: 56 - DNA, 549 bp, homo sapiens
tatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagtttaa

SEQ ID NO: 57 - DNA, 549 bp, homo sapiens
tatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagacgccattagaaactgcatcattggtaaaggaggcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagtttaa

SEQ ID NO: 58 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDAIRNCIIGKGGSYKGTVSITKSGIKCQPWSSMIPHEHSFLPSSYRGKDLQENYCRNPRGEEGGPWCFTSNPEVRYEVCDIPQCSEV

SEQ ID NO: 59 - protein, 182 aa, Artificial Sequence
  Human mutated peptide
YAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDXIRNCIIGKGXSYKGTVSITKSGIKCQPWSSMIPHEHSFLPSSYRGKDLQENYCRNPRGEEGGPWCFTSNPEVRYEVCDIPQCSEV

(19) United States
(12) Patent Application Publication
(10) Pub. No.: US 2009/0215686 A1
(43) Pub. Date: Aug. 27, 2009

(54) NK1-BASED POLYPEPTIDES AND RELATED METHODS

(76) Inventors: Huaqiang Eric Xu, Grand Rapids, MI (US); Ermanno Gherardi, Cambourne (GB)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/043,088

(22) Filed: Mar. 5, 2008

Related U.S. Application Data
(60) Provisional application No. 60/893,032, filed on Mar. 5, 2007.
(60) Provisional application No. 60/969,637, filed on Sep. 2, 2007.
(60) Provisional application No. 60/981,952, filed on Oct. 23, 2007.

(51) Int. Cl. A61K 38/27; C07K 14/475; C12N 5/00; G01N 33/566; C12N 15/18; C12N 15/85; C12N 5/10; A61P 35/00

(52) U.S. Cl. 514/12; 530/399; 435/375; 435/7.1; 536/23.51; 435/320.1; 435/325; 530/405

(57) ABSTRACT

The present invention includes various NK1-based polypeptides and polynucleotides, related compositions, methods of modulating Met activity in a cell, and related methods of treatment. Also, the present invention includes a method for designing an antagonist of a receptor tyrosine kinase from a receptor tyrosine kinase agonist.

### BACKGROUND OF THE INVENTION

Hepatocyte growth factor (HGF, also known as scatter factor) and its receptor Met, a receptor tyrosine kinase (RTK), mediate a network of signaling pathways that control cell proliferation, survival, and motility (Birchmeier et al., 2003). Proper signaling of HGF-Met is essential for normal embryonic development and organ formation (Bladt et al., 1995; Schmidt et al., 1995). In the adult, HGF-Met signaling is involved in angiogenesis, wound healing, and liver regeneration (Borowiak et al., 2004; Huh et al., 2004; Zhang et al., 2003). Besides these normal physiological functions, aberrant activation of HGF/Met signaling has been closely associated with tumor growth, invasion, and metastasis. For example, Met activation by over-expression of the receptor is found in more than 50% of solid tumors and its hyper-activation is generally associated with poor prognosis (http://www.vai.org/met/). Thus, anti-cancer therapy based on Met antagonists has emerged as a prominent and rational goal of pharmaceutical research and development.

HGF is synthesized as an inactive, 90 kDa single chain precursor that is cleaved to form the mature growth factor consisting of two chains linked by a disulfide bond (Bottaro et al., 1991; Gherardi et al., 1989; Mars et al., 1993; Nakamura et al., 1989). The N-terminal α-chain (69 kDa) contains an N-terminal domain (N) followed by four repeats of the kringle domain (K1-K4); the C-terminal β-chain (34 kDa) contains a serine protease-homology (SPH) domain devoid of protease activity (FIG. 1A). NK1 is a fragment containing only the N-terminal domain and the first kringle domain (K1) that occurs naturally through alternative splicing of the primary HGF transcript (Cioce et al., 1996).

NK1 binds Met and is described either as a receptor antagonist or agonist in vitro depending on the context of assay formats and cell types (Lokker and Godowski, 1993; Lokker et al., 1994; Montesano et al., 1998). In vivo studies in transgenic mice, however, have clearly established that NK1 is a potent Met activator (Jakubczak et al., 1998). Despite the fact that the binding affinity of NK1 for Met is 5-20 fold weaker than that of HGF, the ability of NK1 to induce Met dimerization is readily observed and consistent with its agonistic activity in transgenic mice and in cell based assays (Catlow et al., 2003; Jakubczak et al., 1998; Lyon et al., 2004; Rubin et al., 2001; Schwall et al., 1996); and it has been clarified that the agonist activity of NK1 is dependent on the presence of glycosaminoglycans such as heparan sulfate (Catlow et al., 2003; Lyon et al., 2004; Rubin et al., 2001; Schwall et al., 1996). Whereas the detailed interactions between HGF and Met remain poorly characterized, mutagenesis data have pointed out that the fragment corresponding to NK1 is responsible for the high-affinity binding of HGF to Met (Holmes et al., 2007; Lokker and Godowski, 1993; Lokker et al., 1994). Recent mutagenesis and crystallographic data, however, also indicate that the C-terminal β-chain also binds directly to Met and is essential for the agonist activity of the full-length HGF (Stamos et al., 2004). Amino acid sequences for NK1 in various species are available. The human NK1 amino acid sequence is provided in the sequence listing at SEQ ID NO:1. The mouse NK1 amino acid sequence is provided in the sequence listing at SEQ ID NO:2. The PDB code for the mouse and human NK1 is 2QJ4 and 2QJ2, respectively.

Met, the receptor for HGF, was originally identified in a chemically transformed cell line as an oncogene produced by chromosomal rearrangement (Cooper et al., 1984). The 170 kD receptor encoded by the c-Met proto-oncogene contains a large extracellular domain (ectodomain), a transmembrane domain, and an intracellular tyrosine kinase domain (FIG. 1A). The Met ectodomain represents approximately 900 amino acids (amino acids 25-932) that is cleaved into two chains between residues 307 and 308 by the furin protease (Komada et al., 1993). The first 514 amino acids of Met form the so-called sema domain, a 7 bladed β-propeller structure tightly linked with a cysteine-rich domain (amino acids 519-561) that are shared among the semaphorin and plexin protein families (Gherardi et al., 2003; Stamos et al., 2004). The sema domain contains the major binding site for HGF and is required for HGF-induced Met dimerization (Gherardi et al., 2003; Kong-Beltran et al., 2004). Following the sema domain is a stalk structure formed by four tandem repeats of immunoglobulin-like domains.

Met is thought to be activated by HGF through ligand-induced receptor dimerization and there is evidence that HGF can form a complex with 2:2 stoichiometry with a fragment of the Met ectodomain corresponding to the sema and cystine-rich domain (Gherardi et al., 2006). Furthermore, there is evidence that Met on the cell surface is readily cross-linked into dimers or higher order oligomers upon treatment with HGF (Gherardi et al., 2006; Kong-Beltran et al., 2004). Activation of Met was shown to be mediated through receptor dimerization as first demonstrated by the fusion protein of the Met kinase domain with a TRP leucine zipper dimerization motif (Park et al., 1986). However, the mechanism of HGF-induced Met dimerization remains unclear as the isolated full-length Met ectodomain in complex with HGF is predominately monomeric in solution (Gherardi et al., 2003). Recent data shows that the Met sema domain is sufficient for the binding of HGF (Gherardi et al., 2003), and is required for receptor dimerization and activation (Kong-Beltran et al., 2004; Michieli et al., 2004).

Because aberrant activation of the HGF-Met signaling is closely correlated with tumor proliferation, progression, invasion, and metastasis (Birchmeier et al., 2003), targeting Met activation has become an intense area of anti-cancer therapeutical research. Current methods of Met inhibition include small molecules targeting the Met intracellular kinase domain (Christensen et al., 2003), antibodies against HGF or Met (Cao et al., 2001; Petrelli et al., 2006), decoy receptors using the Met extracellular domains (Kong-Beltran et al., 2004; Michieli et al., 2004), and HGF based Met antagonists like single chain HGF derivatives (Matsumoto and Nakamura, 2003; Mazzone et al., 2004). While these methods demonstrate various degrees of Met inhibition, they have displayed limitations with respect to their applications e.g. limited bioavailability and specificity of small molecule inhibitors, the large size and costly production of antibody and decoy Met receptors, protein stability and mixed agonist/antagonist properties of single chain HGF derivatives.

Although receptor dimerization is a general and accepted paradigm for activation of RTKs (Weiss and Schlessinger, 1998), diverse structural mechanisms of ligand-induced receptor dimerization have been revealed by crystallographic studies of ligand-receptor complexes of RTKs (Schlessinger, 2002). Dimerization of the receptors for vascular endothelial growth factor (VEGF) and nerve growth factor (NGF) is mediated by preformed dimeric ligands (Wiesmann et al., 1997; Wiesmann et al., 1999). In FGFR, heparin drives dimerization of FGF ligand and the receptor (Pellegrini et al., 2000; Schlessinger et al., 2000). In contrast, dimerization of EGFR (epidermal growth factor receptor) is through the receptor itself, independent of direct ligand-ligand interactions (Cho and Leahy, 2002; Ferguson et al., 2003; Garrett et al., 2002; Ogiso et al., 2002). The EGF binding site in the receptor is distinct from the receptor dimer interface but the binding of EGF induces a conformational change of the receptor to expose the dimerization loop, thus enabling the receptor to dimerize (Schlessinger, 2002).

### SUMMARY OF THE INVENTION

The present invention includes NK1-based polypeptides comprising the amino acids of any of SEQ ID NO: 8-53, 58, or 59 or SEQ ID NO: 8-53, 58, or 59 with one or more conservative amino acid substitutions, wherein the polypeptide modulates Met activity as compared to wild type NK1; and derivatives thereof. Further, the present invention includes an NK1-based polypeptide comprising the amino acids of any of SEQ ID NO: 8-13, 20-36, 58, and 59 wherein the polypeptide is a Met antagonist; and derivatives thereof. Also, the present invention includes an NK1-based polypeptide comprising amino acids of any of SEQ ID NO: 54 or 55, or SEQ ID NO: 54 or 55 with one or more conservative amino acid substitutions; and derivatives thereof.

In another embodiment, the present inventions include: (1) an NK1-based polypeptide comprising amino acids of SEQ ID NO: 1 wherein the amino acid at one of wild type NK1 amino acid positions 77, 82, 83, 85, 122, 123, 124, 126, 127, 134, 137, 139, 140, 141, and 142 is substituted with an amino acid not normally found at that position in SEQ ID NO: 1, and wherein the polypeptide decreases Met activity as compared to wild type NK1, and derivatives thereof, (2) an NK1-based polypeptide comprising amino acids of SEQ ID NO: 1 wherein the amino acid at two of wild type NK1 amino acid positions 77, 82, 83, 85, 122, 123, 124, 126, 134, 137, 139, 141 and 142 is substituted with an amino acid not normally found at that position in SEQ ID NO: 1, and wherein the polypeptide decreases Met activity as compared to wild type NK1, and derivatives thereof, and (3) an NK1-based polypeptide comprising amino acids of SEQ ID NO: 1 wherein the amino acid at three of wild type NK1 amino acid positions 77, 83, 85, 122, 123, 124, 126, 127, 134, 137, 139, 140, 141, and 142 is substituted with an amino acid not normally found at that position in SEQ ID NO: 1, and wherein the polypeptide decreases Met activity as compared to wild type NK1, and derivatives thereof.

Also included is a composition comprising any of the above-described polypeptides and a pharmaceutically acceptable diluent or carrier.

A method of the present invention includes reducing Met activity in a cell comprising introducing the above-described polypeptides to the cell, e.g., a tumor cell. Another method includes increasing Met activity in a cell by introducing to the cell an NK1-based polypeptide comprising amino acids of any of SEQ ID NO: 54 or 55, or SEQ ID NO: 54 or 55 with one or more conservative amino acid substitutions; and a derivative thereof.

A further method includes designing an antagonist by converting a growth factor from a receptor tyrosine kinase agonist to an antagonist, wherein the native receptor dimerizes or oligomerizes to become active, comprising the steps: identifying a region of said growth factor that promotes said dimerization or oligomerization of said receptor; providing growth factor mutants in which at least one amino acid in said region of said growth factor that promotes said dimerization or oligomerization has been substituted with another amino acid; screening said mutants to determine receptor dimerization or oligomerization activity and to determine the binding affinity of the mutants to the receptor, and selecting one or more mutants that have a decreased ability as compared to wild type growth factor to promote dimerization or oligomerization of the receptor but retain the ability to bind the receptor. The receptor tyrosine kinase of interest may be, for example, Met, epidermal growth factor receptor (EGFR), nerve growth factor receptor (NGFR), vascular endothelial growth factor receptor (VEGFR), fibroblast growth factor receptor (FGFR), stem cell factor receptor, and macrophage-stimulating protein receptor (RON). With this method, a proximity assay may be used to determine receptor dimerization or oligomerization activity or to determine the binding affinity of said mutants to the receptor.

The present inventions also include a polynucleotide coding for any one of the above-described polypeptides or derivatives; an expression vector comprising such polynucleotide or derivative operably linked to a promoter; and a host cell carrying the vector.

An additional method includes treating a patient in need of control of cell growth, cell proliferation, cell survival, or cell motility, said method comprising administering to said patient a therapeutically effective amount of one of the polypeptides or derivatives described above. Also, one method includes treating a patient in need of anti-cancer therapy, said method comprising administering to said patient a therapeutically effective amount of one of the above-described polypeptides or derivatives which acts as a Met antagonist.

A kit is also provided herein and includes a therapeutic dose of bioactive agent for use in treating a patient, comprising: a container; and a volume of material stored within the container in substantially sterile form; wherein the material comprises any one of the above-described polypeptides or a derivative thereof.

A further method of the present invention includes treating an abnormal cellular proliferation condition associated with a region of tissue in a living patient, comprising: delivering a therapeutically effective amount of a material to the region of tissue; inhibiting the abnormal cellular proliferation condition with the therapeutic amount of the material; wherein the material comprises any one of the above-described polypeptides or derivatives which acts as a Met antagonist. Alternatively, with this method, the region of tissue comprises a tumor, and the abnormal cellular proliferation condition comprises cancer, and further; the therapeutic amount of the material is delivered to the tumor; and proliferation of the cancer is inhibited with the therapeutic dose of the material.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown. Drawings are not necessary to scale. Certain features of the invention may be exaggerated in scale or shown in schematic form in the interest of clarity and conciseness.

FIGS. 1A-E show the binding of human and mouse NK1 to Met. FIG. 1A is a schematic representation of the domain arrangement of HGF and Met. FIG. 1B shows the purified proteins used in the examples. Proteins shown are the mouse and human NK1 (lanes 1 and 2), the human NK1 with R134G mutation (lane 3), biotinylated human NK1 (lane 4), and the Met sema domain (lane 5). The star (*) in lane 2 indicates truncated N domain of human NK1. The dashed line indicates that lanes 4-5 are run in a different gel from other lanes. FIG. 1C is a diagram of AlphaScreen assay for detecting NK1-Met interactions. FIG. 1D is a graph showing binding of biotinylated NK1 to Met in the absence or presence of heparin as measured by AlphaScreen assay. FIG. 1E is a graph showing binding affinity (IC50 values) of various NK1s to Met as determined by the inhibition of the binding of the biotinylated NK1 to Met with dose competition of unlabeled NK1s.

FIGS. 2A-D show Met dimerization induced by NK1 binding. FIG. 2A is a diagram of the AlphaScreen assay for detecting Met dimerization promoted by NK1 binding. FIG. 2B is a graph showing Met dimerization induced by 1 μM of NK1 in the presence and absence of 1 μM heparin as measured by AlphaScreen assays. FIG. 2C are dose curves of various NK1 that induce Met dimerization in the presence of 1 μM heparin. FIG. 2D shows NK1-induced Met dimerization as determined by dynamic light scattering. Except for the NK1-Met without heparin, all measurements have SOS values less than 1.0 indicating mono-dispersion of the complex. The high SOS value in the NK1-Met without heparin indicate a poly-dispersed complex, reflecting the unstable nature of the Met-NK1 complex in the absence of heparin.

FIGS. 3A-E are crystal structures of the mouse and human NK1. FIG. 3A is an overall view of the head-to-tail dimer of the mouse NK1. The letters (K, D, Y, and N) indicate the position of residues K85, D123, Y124 and N127. Only one NK1 monomer is labeled. FIG. 3B shows the NK1 dimer interface and the intermolecular interactions. The list of interactions includes non H-bond packing and H-bond interactions within 4.0 Å between the two NK1 monomers. FIG. 3C shows interactions mediated by Y124 and K85 with V140, D202, I203, and P204. Hydrogen bonds are indicated by dashed lines. FIG. 3D shows interactions mediated by K122, D123 and N127 with K122, D123 and N127. Hydrogen bonds are indicated by dashed lines. Double-head arrows indicate reciprocal interactions between two monomers and single-head arrows indicate interactions from one monomer to the other monomer. FIG. 3E shows a comparison of the mouse and human NK1 (superposition of the Cα atoms). Arrows indicate the heparin binding sites identified in previous studies (Lietha et al., 2001; Zhou et al., 1999).

FIG. 3F is a sequence alignment of NK1 from various species: specifically; human “wild type” NK1 (SEQ ID NO: 1), mouse NK1 (SEQ ID NO: 2), rat NK1 (SEQ ID NO: 3), cow NK1 (SEQ ID NO: 4), cat NK1 (SEQ ID NO: 5), dog NK1 (SEQ ID NO: 6), and chicken NK1 (SEQ ID NO: 7). SEQ ID NO: 56 shows the cDNA sequence for encoding human NK1. Note: for each of the amino acid sequences identified in the concurrently filed Sequence Listing, wild type NK1 amino acid position 28 is shown as the first amino acid in the sequence (e.g., in SEQ ID NO.1, the first amino acid “tyrosine” is the amino acid located at position 28 of wild type NK1, the second amino acid “alanine” is the amino acid located at position 29 of wild type NK1, etc.). The secondary structure of the mouse and human NK1 is underlined. Residues involved in NK1 dimer formation and heparin binding are marked with stars and H, respectively, and residues involved in formation of the five disulfide bonds are marked with A, B, C, D, and E. The arrow indicates position of R134.

FIGS. 4A-C show the effects of the NK1 dimerization mutants. FIG. 4A is a graph showing the ability of various NK1 mutants to induce Met dimerization in the presence and absence of 1.0 μM heparin. FIG. 4B shows the binding affinity (IC₅₀ values) of NK1 mutants to Met as determined by competition of the binding of the biotinylated NK1 to Met. FIG. 4C is a graph showing the ability of NK1 mutants to inhibit NK1-mediated Met dimerization.

FIGS. 5A-E show the effects of NK1 dimerization mutants in cell-based assays. FIG. 5A is a graph showing the effects of NK1 mutants (1 μM) on uPA assays in the presence and absence of HGF. FIG. 5B is a graph showing the effects of NK1 mutants (1 μM) on proliferation of MDCK cells in the presence and absence of HGF. FIG. 5C shows the effects of NK1 (1 μM) on MDCK cell scattering assays in the presence and absence of HGF. FIG. 5D shows the effects of NK1 mutants (1 μM) on branching morphogenesis of the prostate cancer cell line DU145. Arrows indicate the cell branching induced by HGF. FIG. 5E is a graph showing the effects of NK1 mutants (1 μM) on invasion of the glioblastoma cell line DBTRG. All assays were performed with 60 ng/ml of HGF when indicated and 1 μM of heparin except for the control in FIG. 5A.

FIGS. 6A-B are models of Met activation and inhibition by NK1 and its mutants. FIG. 6A is a diagram showing the mechanism of Met binding and dimerization by NK1. FIG. 6B is a diagram showing the mechanism of Met binding and inhibition by NK1 mutants. The arrowheads indicate NK1 mutations that disrupt the NK1 dimer interface.

FIGS. 7A-C show the functional properties of the V140R NK1 mutant. FIG. 7A is a graph showing a comparison of the ability to induce Met dimerization by various NK1s in the presence and absence of 1.0 μM of heparin. FIG. 7B is a graph showing a comparison of the ability of various NK1 mutants to activate uPA activity in MDCK cells in the presence and absence of 60 ng/ml of HGF. Control is buffer only, and heparin is the presence of 1.0 μM heparin in the buffer. All assays for NK1s are performed with heparin. FIG. 7C shows the ability of various NK1s to induce and inhibit scattering of MDCK cells in the presence and absence of 60 ng/ml of HGF. All assays are performed in the presence of 1.0 μM heparin.

FIGS. 8A-8D show dimerization-deficient mutants of HGF/SF. FIG. 8 A shows the domain structure of HGF/SF (top) and NK1 (bottom). N: N-terminal, K: kringle, SPH: serine proteinase homology. FIG. 8B shows the surface representation of the NK1 protomer (pdb accession: 1NK1). Selected residues involved in dimer formation are labelled and coloured in yellow. Areas coloured in blue and red are located on different faces of the NK1 protomer and represent patches of residues involved in MET or heparin binding respectively. FIG. 8C shows HGF/SF mutants studied by the inventors: mutations at the dimer interface are listed in black, other mutations are shown in blue. FIG. 8D shows MET binding (left columns) and biological activity (right columns) of wild-type (wt) HGF/SF and mutants. Mutations at the dimer interface are shown in yellow, other mutations in blue.

FIGS. 9A-9G show MET-binding and dimerization activity of NK1 mutants. FIGS. 9A-9D show binding of: wild-type NK1, the LC(Y124A) linker mutant, the LE (N127A) linker mutant and, the K1 mutant K1D (V140E:T142A) to MET567 by surface plasmon resonance, respectively. For each protein, two-fold dilutions were used from a starting concentration of 200 nM. FIGS. 9E-9G show stoichiometries of the complexes formed in solution by wild type NK1 and the linker mutant LC and MET567 by small angle X-ray scattering. FIG. 9E shows scattering intensities as functions of momentum transfer (s=4π sin(θ)/λ, where 2θ is the scattering angle and λ=0.15 nm is the X-ray wavelength). Experimental data are shown as black dots, fits from ab initio models are presented by red solid lines. FIGS. 9F and 9G are ab initio models of the complexes formed by the LC (Y124A) linker mutant and wild-type NK1 (yellow beads) and MET567 (grey beads) in the presence of 12mer heparin. The complex formed by the LC mutant has 1:1 stoichiometry in contrast to the one formed by wild-type NK1 which has 2:2 stoichiometry.

FIGS. 10A-10V show receptor agonistic activity of the NK1 mutants. FIGS. 10A-10T show MDCK colony scatter assays: standard medium (FIG. 10A); 10-11 M HGF/SF (FIG. 10B); LC, LE, LF, CH linker and/or K1 mutants at the dimer interface (FIGS. 10C-10E, 10F-10H, 10I-10K, and 10L-10N, respectively); and K1M and K1N mutants at the Met binding interface (FIGS. 10O-10-Q and 10R-10T, respectively). The activity of all mutants is shown at three concentrations (10-9, 10-8 and 10-7 M) in the order given. FIG. 10U is a bar graph showing DNA synthesis in MK cells in response to wild-type NK 1, linker and/or kringle 1 mutants at the dimerization interface and (LC, LE, LF, and CH) and NK4. The asterisks show statistically significant differences compared to basal medium (*=p<0.5, **=p<0.01, ***=p<0.001). FIG. 10V is a blot showing Erk1/2 phosphorylation in MK cells in response to NK1 and selected linker and/or kringle mutants at the dimerization interface (LE, K1D and K1 G). The concentrations at which proteins were tested are indicated below the bar chart or the blot.

FIGS. 11A-11L show receptor antagonistic activity of the NK1 mutants. FIGS. 11A-11J MDCK colony scatter assays: standard medium (FIG. 11A); 3×10-10 M HGF/SF (FIG. 11B); 3×10-10 M HGF/SF+10-8 M or 10-7 M concentrations of NK1 (FIGS. 11C and 11D); 3×10-10 M HGF/SF, +10-8 M or 10-7 M concentrations of the K1N mutant (FIGS. 11E and 11F); 3×10-10 M HGF/SF, +10-8 M or 10-7 M concentrations of the linker mutant LE (FIGS. 11G and 11H); and 3×10-10 M HGF/SF, +10-8 M or 10-7 M concentrations of NK4 (FIGS. 11I and 11J). FIG. 11K is a bar graph showing DNA synthesis in MK cells in the presence of 10-10 M HGF/SF and the concentrations indicated of NK1, the linker or kringle 1 mutants LC, LE, LF, LG, K1D, K1G and NK4. The asterisks show statistically significant differences compared to well incubated with 10-10 M HGF/SF alone (*=p<0.5, **=p<0.01, ***=p<0.001). FIG. 11L is a blot showing Erk1/2 phosphorylation in response to 10-10 M HGF/SF (+ve) or 10-10 M HGF/SF plus the concentrations indicated of NK1, selected linker or kringle 1 mutants or NK4. The concentrations at which proteins were tested are indicated below the bar chart or the blot.

FIG. 12 is graph showing binding of 1×10-9 biotin-labeled HGF/SF to MET928 in the presence of the concentrations shown of unlabeled HGF/SF or mutants ND, NF, LH, K1H and K1N.

FIG. 13 is a bar graph showing the effect of NK1 mutants on Met dimerization.

### CROSS-REFERENCE TO RELATED APPLICATIONS

This application claims the benefit of the following provisional applications: Ser. No. 60/893,032 filed Mar. 5, 2007, entitled A MECHANISTIC BASIS FOR CONVERTING A GROWTH-FACTOR RECEPTOR AGONIST TO AN ANTAGONIST; Ser. No. 60/969,637 filed Sep. 2, 2007, entitled NK-1 BASED POLYPEPTIDES AND RELATED METHODS; and Ser. No. 60/981,952 filed Oct. 23, 2007, entitled NK-1 BASED POLYPEPTIDES AND RELATED METHODS, the entire contents of which are incorporated herein in their entirety.

### DETAILED DESCRIPTION OF THE PREFERRED EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention.

The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples and Sequence Listing included hereafter.

For clarity of disclosure, and not by way of limitation, the detailed description of the invention is divided into the subsections that follow.

The text file filed concurrently with this application, titled “VAN67P392.txt” contains material identified as SEQ ID NO: 1-59, which material is incorporated herein by reference. This text file was created on Mar. 5, 2008, and is 109,775 bytes.

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. Although any methods, devices and materials similar or equivalent to those described herein can be used in the practice or testing of the invention, the preferred methods, devices and materials are now described.

In this specification and the appended claims, the singular forms “a,” “an” and “the” include plural reference unless the context clearly dictates otherwise.

The term “amino acid” refers to naturally occurring and synthetic amino acids, as well as amino acid analogues and amino acid mimetics that function in a manner similar to the naturally occurring amino acids. Naturally occurring amino acids are those encoded by the genetic code, as well as those amino acids that are later modified, e.g., hydroxyproline, alpha-carboxyglutamate, and O-phosphoserine. Amino acid analogues refers to compounds that have the same basic chemical structure as a naturally occurring amino acid, i.e., an alpha carbon that is bound to a hydrogen, a carboxyl group, an amino group, and an R group (e.g., homoserine, norleucine, methionine sulfoxide, methionine methyl sulfonium). Such analogues have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “cell” is used in its usual biological sense, and does not refer to an entire multicellular organism. The cell can, for example, be in vitro, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

A polypeptide that “modulates” Met activity either activates (promotes, enhances, increases) or inhibits (suppresses, blocks, decreases) Met activity and such modulation is identified by in vitro or in vivo assays of this activity or downstream activities, some of which assays are described hereinbelow.

“Nucleic acid” refers to deoxyribonucleotides or ribonucleotides and polymers thereof in either single- or double-stranded form. The term encompasses nucleic acids containing known nucleotide analogues or modified backbone residues or linkages, which are synthetic, naturally occurring, and non-naturally occurring, which have similar binding properties as the reference nucleic acid, and which are metabolized in a manner similar to the reference nucleotides. Examples of such analogues include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses variants thereof (e.g., degenerate codon substitutions that encode the same amino acids) and complementary sequences, as well as the sequence explicitly indicated. The term “nucleic acid” is used interchangeably with gene, cDNA, mRNA, oligonucleotide, and polynucleotide.

The term “operably linked” refers to a juxtaposition wherein the components described are in a relationship permitting them to function in their intended manner. A control sequence “operably linked” to a coding sequence is ligated in such a way that expression of the coding sequence is achieved under condition compatible with the control sequences.

“Patient” refers to a mammal, preferably a human, in need of treatment for a condition, disorder or disease.

The terms “polypeptide,” “peptide,” and “protein” are used interchangeably herein to refer to a polymer of amino acid residues. These terms also include amino acid polymers in which one or more amino acid residue is an analogue or mimetic of a corresponding naturally occurring amino acid, as well as to naturally occurring amino acid polymers.

By “treatment” or “treating” it will be understood that this refers to any administration of a polypeptide intended to alleviate the severity of a disease being treated, to provide relief from the symptoms of the disease or to prevent or slow down the development of the disease in an individual with a disease condition or at risk of developing the disease condition.

By “therapeutically effective amount” is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “tumor cell” refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. A “tumor” includes at least one tumor cell.

Here disclosed is a mechanistic basis for designing Met antagonists based on NK1, a natural variant of HGF containing the N-terminal and the first kringle domain of HGF. Through detailed biochemical and structural analyses, it was determined that both mouse and human NK1 induce Met dimerization via a conserved NK1-NK1 dimer interface. Both the mouse and human NK1 are able to bind and induce Met dimerization, and novel crystal structures of these two proteins reveal a conserved NK1-NK1 dimer interface. Mutations in the NK1-NK1 dimer interface abolish its ability to promote Met dimerization but retain full Met-binding activity. Importantly, by introducing mutations in the NK1 dimer interface it was found that the Met binding capability of NK1 can be separated from its ability to promote Met dimerization and activation. These NK1 dimer-interface mutants retain full Met binding ability, but inhibit HGF-mediated Met activation. Further, the disclosed NK1 mutants decrease or reduce Met activity (i.e., they act as Met antagonists) by inhibiting HGF-mediated cell scattering, proliferation, branching and invasion.

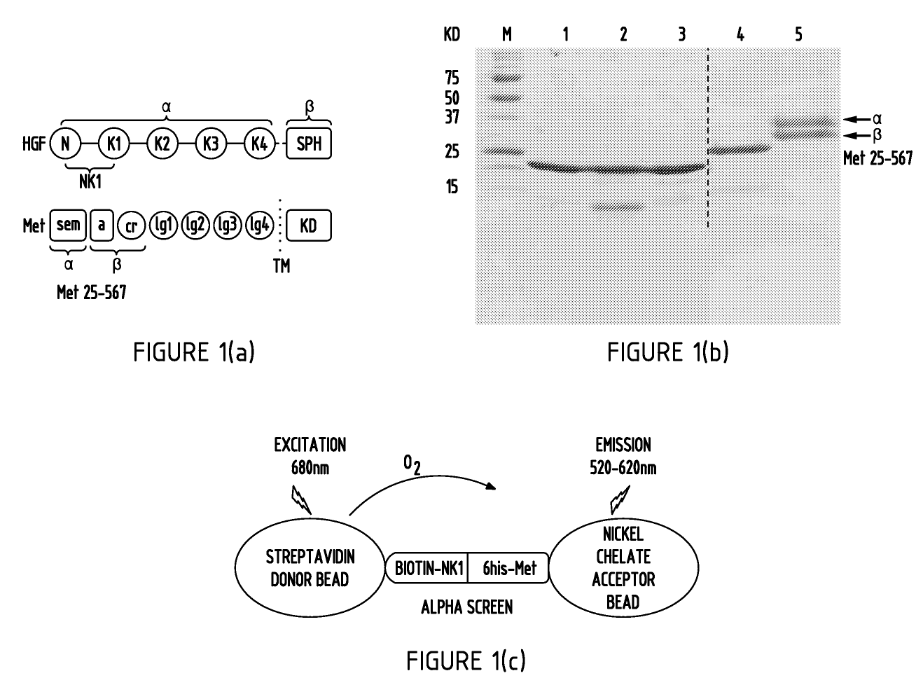

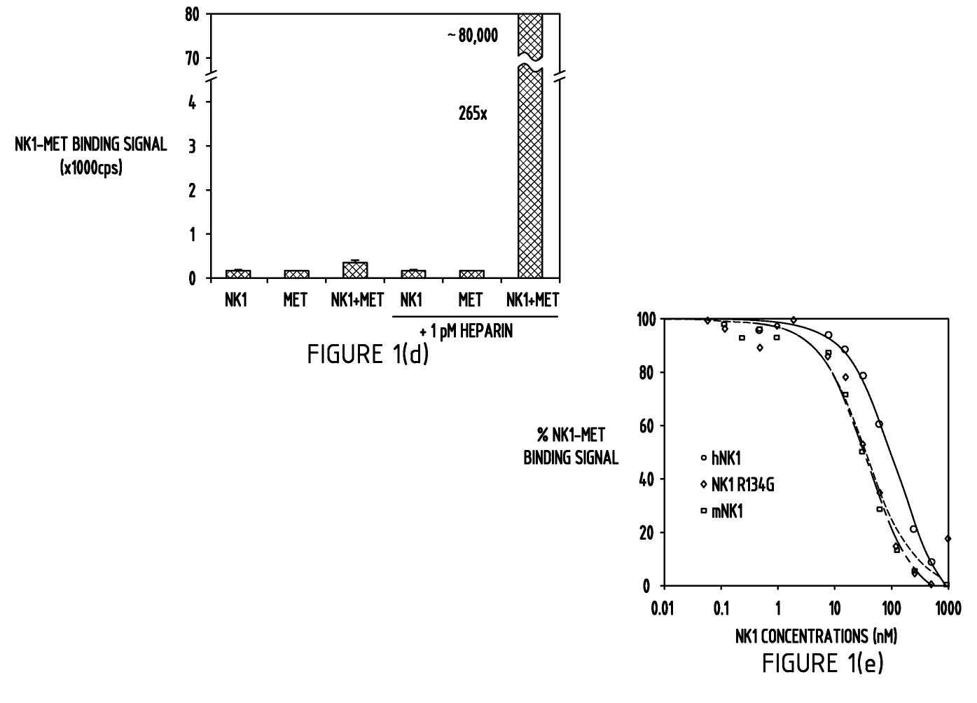

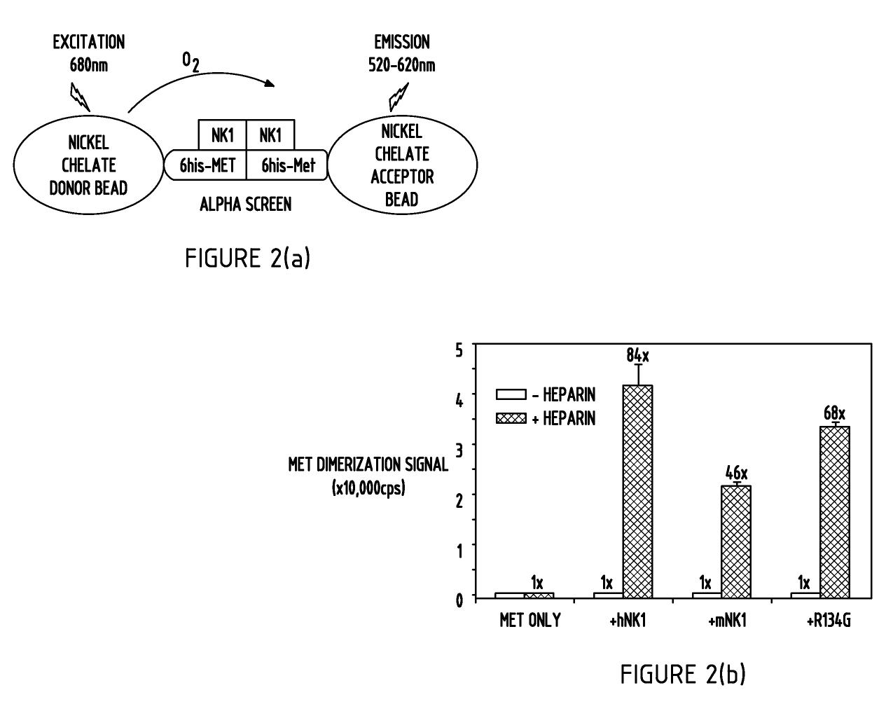

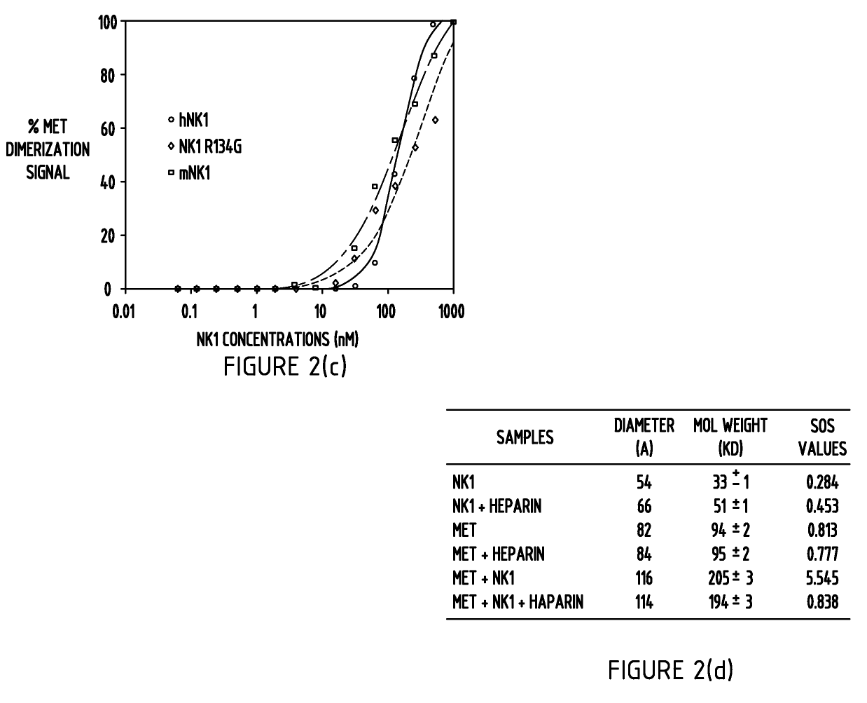

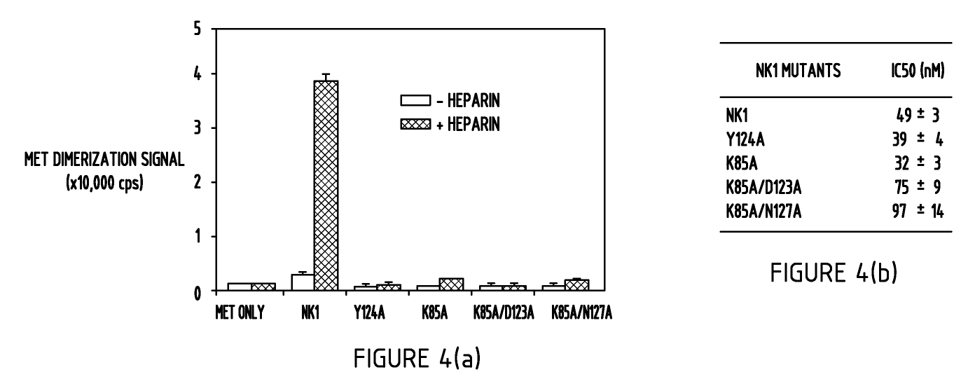

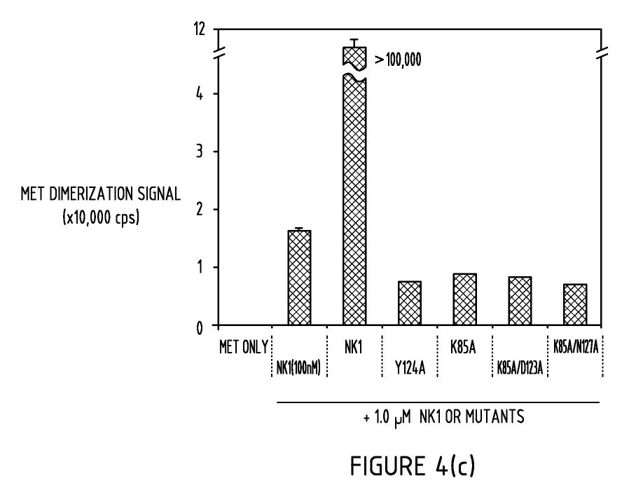

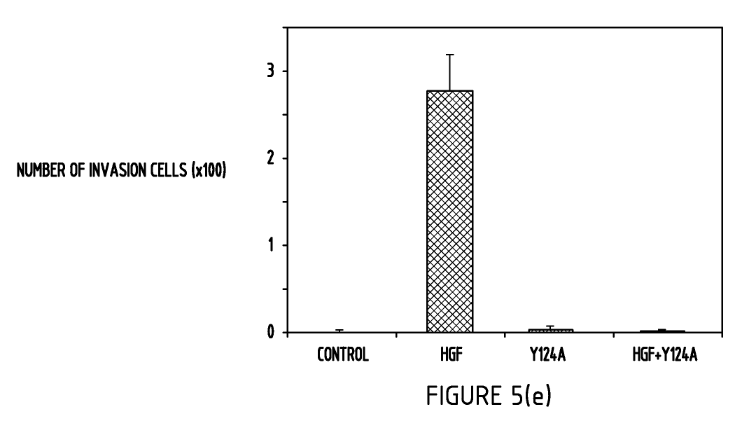

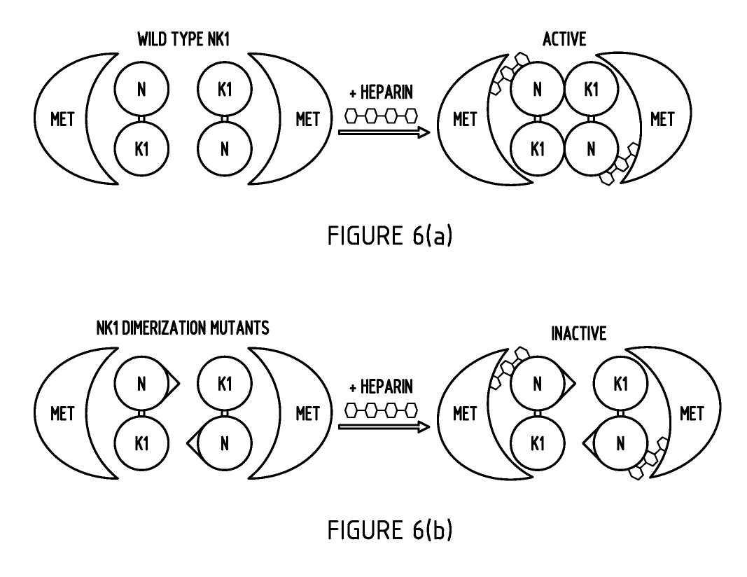

While not being bound by any particular theory, one plausible mechanism for NK1 to induce Met dimerization is that dimerization of Met is mediated by the NK1-NK1 dimer interface, in a manner similar to receptor/ligand complexes of VEGF and NGF (FIG. 6A). In this model, NK1 is monomeric in the absence of heparin. Addition of heparin drives NK1 dimerization resulting in the formation of a stable complex with Met and resulting in receptor activation. Although the validation of such a model waits for a structure of the NK1/Met/heparin complex, this model is supported by a number of experimental evidences. The first evidence is that NK1 forms a dimer in a heparin dependent manner, and this is directly correlated with the requirement of heparin for NK1 to bind and promote Met dimerization (FIGS. 1 and 2). The consistent requirement of heparin for NK1 dimerization and for the formation of a stable NK1/Met complex resulting in Met dimerization suggests that the NK1-NK1 dimer plays a direct role in mediating Met dimerization and activation. Secondly, the crystal structures of both mouse and human NK1 reveal a conserved NK1-NK1 interface and mutations that are designed to alter the NK1 dimer interface (Y124A, K85A, K85A/D123A, and K85A/N127A) abolish the ability of NK1 to induce Met dimerization (FIG. 4A), providing strong support that the NK1 dimer interface observed in the crystal structures is critical for Met dimerization and activation. The highly conserved nature of residues involved in the formation of the NK1 dimer interface further indicate that this mode of Met dimerization and activation by NK1 is preserved across species from chicken to man. Finally, the recently determined low resolution structure of a Met/HGF complex by cryo-electron microscopy and small angle X-ray scattering, suggests that the packing of the HGF/Met complex may be mediated by the NK1-NK1 dimer interface (Gherardi et al., 2006) and, interestingly, the K85A and/or N127A mutations in the context of the full-length HGF abolish its ability to scatter MDCK cells, further supporting the importance of the observed NK1 dimer interface in HGF-mediated Met activation (Gherardi et al., 2006).

The above mechanism of Met activation by HGF via NK1 dimerization provides a rational basis for designing an NK1-based Met antagonist by selectively disrupting the ability of NK1 to dimerize (FIG. 6B). Example mutant NK1 antagonists of Met are Y124A, K85A, K85A/D123A, K85A/N127A, and D123A, N127A. Amino acid sequences for these mutant NK1s are provided in the sequence listing at SEQ ID NO: 8-13. SEQ ID NO 58 includes the amino acid sequence for a possible NK1 antagonist R134G/Y124A; SEQ ID NO 57 is the nucleic acid sequence for this polypeptide. With most mutants, the residue has been changed to an alanine, however, it is contemplated that any substituted amino acid not normally found at that position would be acceptable. Such NK1 mutants are Y124X, K85X, K85X/D123X, K85X/N127X, D123X, N127X, and R134X/Y124X where “X” is any substituted amino acid not normally found at the indicated position (SEQ ID NO: 14-19 and 59).

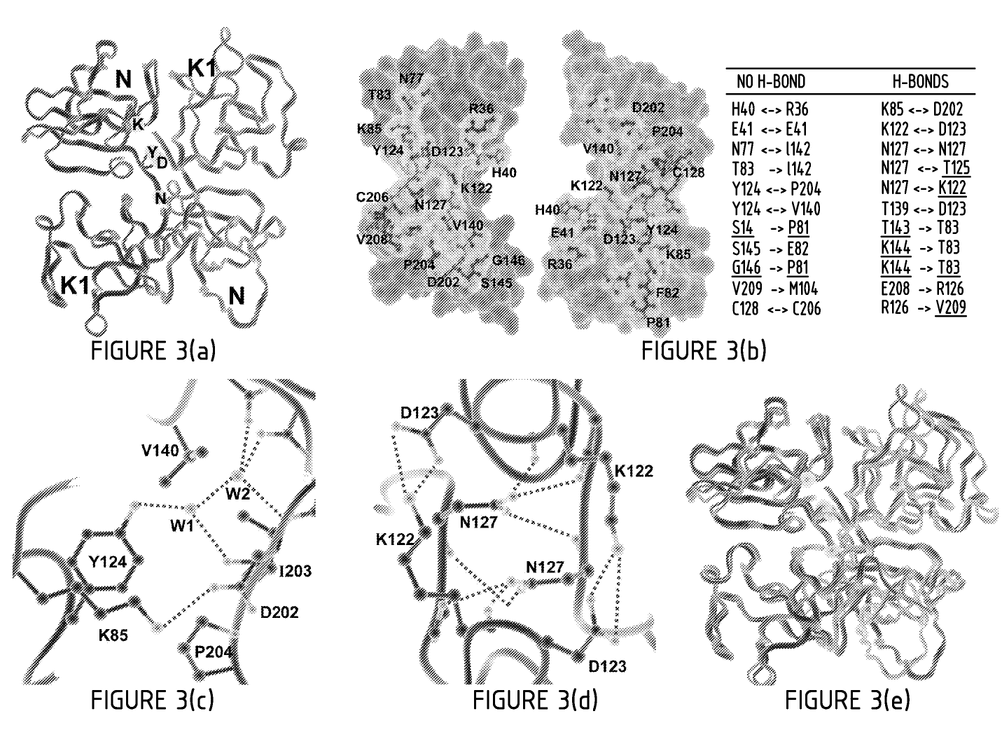

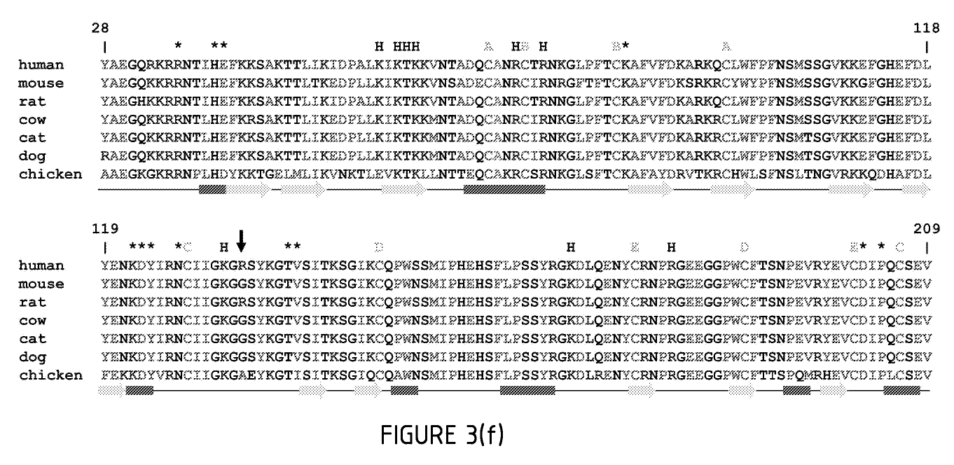

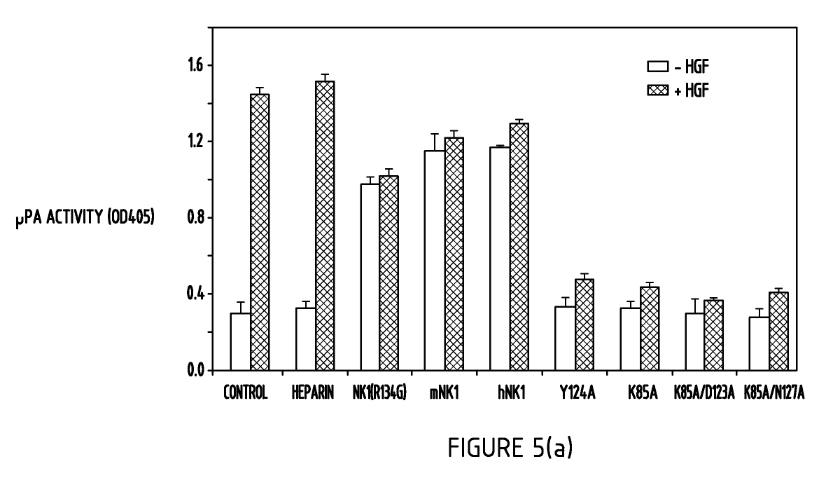

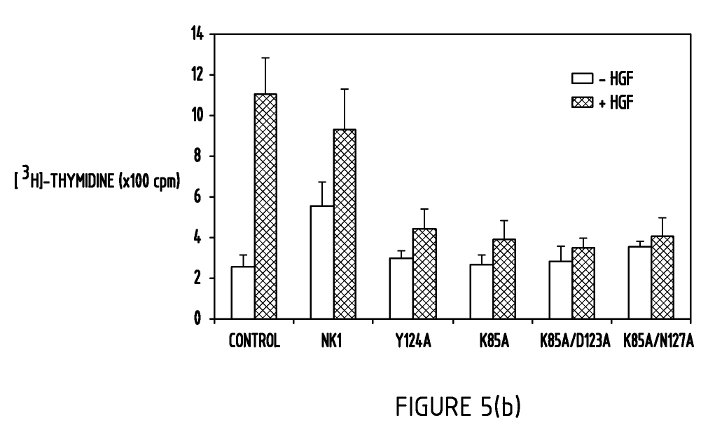

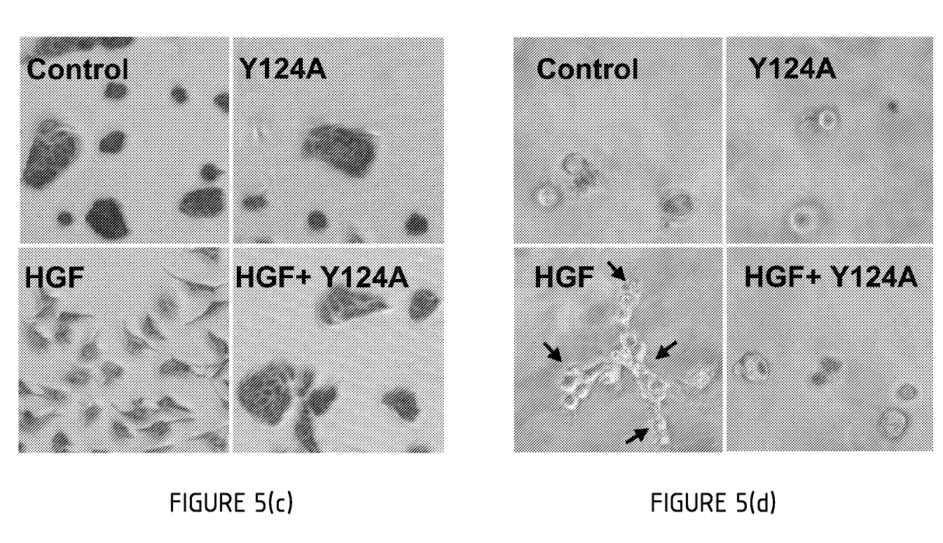

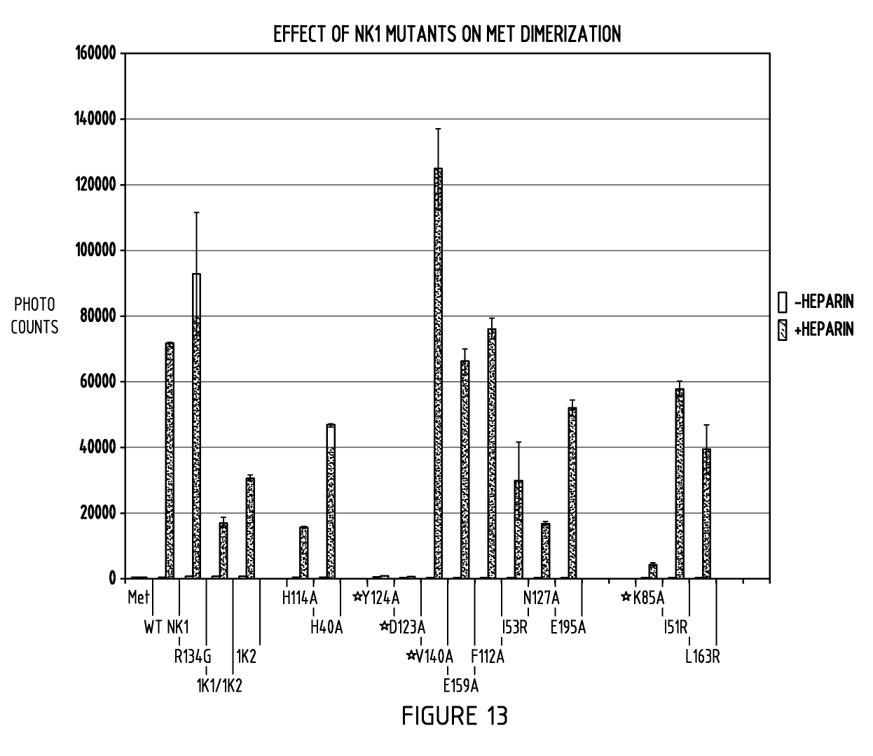

The NK1 mutants shown in FIG. 4B retain full Met-binding ability and they act as effective Met antagonists (FIG. 5). The ability to disrupt NK1-induced Met dimerization by a number of NK1 mutations indicates that receptor dimerization is very sensitive to the perturbation of the NK1 dimer interface, and provides a way to make and design other NK1 dimer mutants to serve as Met antagonists. In fact, disclosed are mutations made in several other residues involved in NK1 dimer formation listed in FIG. 3B. Most of these NK1 dimer mutants lost their ability to induce Met dimerization (data not shown). Also, Met dimerization was disrupted for the NK1 mutants as shown in FIG. 13.

Additional mutants of the NK1 dimer interface are included in Table 1 (SEQ ID NO. 20-36). Mutations in the N domain (N), the linker region (L), kringle 1 (K1), and in combinations thereof (C) are shown in Table 1. These NK1-based polypeptides are Met antagonists, having reduced Met activity, as demonstrated by a MDCK colony scatter assay (See, Example 7 below).

TABLE 1

| | | SEQ |
|---|---|---|
| MUTANT CODE | MUTATION | ID |
| N.B | N77A | 20 |
| N.C | F82A:T83A:K85A | 21 |
| N.D | N77A:F82A:T83A:K85A | 22 |
| L.A | K122A | 23 |
| L.B | D123A | 12 |
| L.C. | Y124A | 8 |
| L.D. | R126A | 24 |
| L.E | N127A | 13 |
| L.F. | D123A:Y124A | 25 |
| L.G | D123A:N127A | 26 |
| L.H | D123A:Y124A:N127A | 27 |
| K1.D | V140A:I142A | 28 |
| K1.F | T139A:S141A:I142A | 29 |
| K1.G | K137A:T139A:S141A:I142A | 30 |
| K1.H | K137A:T139A:V140A:I142A | 31 |
| C.A | F82A:T83A:K85A:D123A:N127A | 32 |
| C.B. | D123A:K137A | 33 |
| C.F | D123A:K137A:T139A:V140A:I142A | 34 |
| C.G | D123A:K137A:T139A:S141A:I142A | 35 |
| C.H | D123A:N127A:K137A:T139A:V140A:I142A | 36 |

With each mutant in Table 1, the residue has been changed to an alanine, however, it is contemplated that any substituted amino acid not normally found at the position of the altered residue would be acceptable. Such NK1 antagonists of Met are N77X; F82X:T83X:K85X; N77X:F82X:T83X:K85X; K122X; R126X; D123X:Y124X; D123X:N127X; D123X:Y124X:N127X; V140X:T142X; T139X:S141X:T142X; K137X:T139X:S141X:I142X; K137X:T139X:V140X:I142X; F82X:T83X:K85X:D123X:N127X; D123X:K137X; D123X:K137X:T139X:V140X:I142X; D123X:K137X:T139X:S141X:I142X; D123X:N127X:K137X:T139X:V140X:I142×, where “X” is any substituted amino acid not normally found at the indicated position (SEQ ID NO: 37-53).

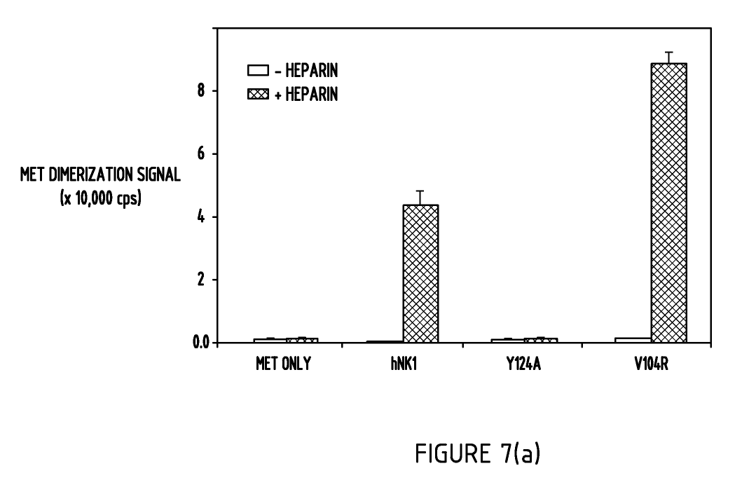

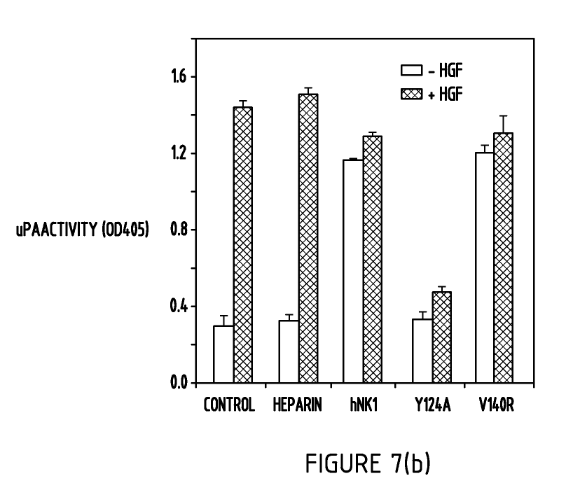

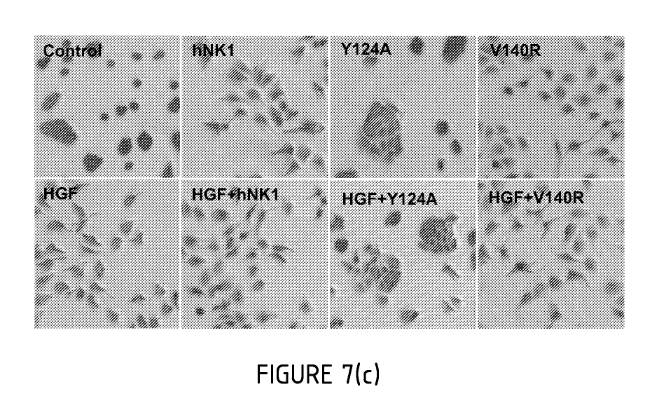

One exception is the V140R mutant (SEQ ID NO: 54), which actually has an increased ability to promote Met dimerization over the wild type NK1 (FIG. 7A). Cell based experiments indicate that the V140R mutant acts as a Met agonist as indicated by induction of uPA activity and scattering of MDCK cells (FIGS. 7B and 7C). In the structure, V140 is loosely packed against Y124 in the NK1 dimer interface (FIG. 3C). An arginine residue can be easily modeled into the V140 position, consistent with the V140R mutant phenotype. The 140 residue has been changed to an arginine, however, it is contemplated that any substituted amino acid not normally found at that position would be acceptable. See, for example, the NK1 agonist V140X, where “X” is any substituted amino acid not normally found at position 140 (SEQ ID NO: 55). The spectrum of the NK1 dimer interface mutants indicates that NK1 dimerization can be tuned according to the purpose of modulating its ability to activate or inhibit Met.

The NK1 based Met antagonists described here have several distinct advantages to known methods of Met inhibition. First, NK1 is a naturally circulated HGF variant that targets the extracellular domain of Met. Second, NK1 is a much smaller protein than an antibody, decoy Met receptor, or single chain HGF and NK4. Third, because of its small size, NK1 can be easily produced in large quantities and high purity. Fourth, the potency of the disclosed NK1-based antagonists is currently within one order of magnitude of HGF and approaching the potency of small molecules or antibodies. Finally, the disclosed antagonists are based on endogenous proteins with small variations, while antibodies are exogenous and may be antigenic.

The present invention includes methods for modulating Met activity in a Met expressing cell as compared to the Met activity from wild type NK1. As described hereinabove, most of the disclosed NK1 mutants are antagonists of Met activity, e.g., the polypeptides of amino acid sequences 8-11. That is, these polypeptides successfully bind Met in competition with wild type NK1, fail to induce Met dimerization, and inhibit Met activity (as shown in the Examples herein). Similarly, the method for modulating Met activity in a Met expressing cell also includes use of an NK1 mutant, or derivative thereof, that is an agonist of Met activity, e.g., the polypeptide of amino acid SEQ ID NO: 54. The method of modulating Met activity includes introducing one of the novel NK1 polypeptides, or a derivative thereof, into a Met-expressing cell. For example, as described below, in one embodiment a polynucleotide encoding one of the novel NK1 polypeptides, or a derivative thereof, is incorporated into a recombinant replicable vector that is used to replicate the polynucleotide in the Met-expressing cell.

Conservative Amino Acid Substitutions of the NK1 Mutants:

In one aspect, the present invention also include a polypeptide having an amino acid sequence of any of SEQ ID NO: 8-59, which polypeptide includes one or more “conservative amino acid substitutions”. Such polypeptide may differ from the original sequence such that it has 90%, 95%, or 98% identity with the amino acid sequence of SEQ ID NO: 8-59. It is well known in the art that the amino acids within the same conservative group can typically substitute for one another without substantially affecting the function of a protein.

Conservative substitution tables providing functionally similar amino acids are well known in the art (see for example Table 2). For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W. H. Freeman and Company (1984); Schultz and Schimer, Principles of Protein Structure, Springer-Verlag (1979)).

One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservative amino acid substitutions” as long as the polypeptide retains its desired properties, i.e., with a conservative substitution in an NK1-based polypeptide antagonist of Met, the polypeptide will bind to Met and inhibit Met activity.

For a detailed description of protein chemistry and structure, see Schulz, G E et al., Principles of Protein Structure, Springer-Verlag, New York, 1978, and Creighton, T. E., Proteins: Structure and Molecular Properties, W.H. Freeman & Co., San Francisco, 1983, which are hereby incorporated by reference. The types of substitutions which may be made in the protein molecule may be based on analysis of the frequencies of amino acid changes between a homologous protein of different species.

Conservative substitution groups are set forth in Table 2 based on shared properties.

TABLE 2
Conservative substitution.

| Original | |
|---|---|
| Residue | Conservative Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

Various conservative substitutions are envisioned to be within the scope of the invention. For instance, it would be within the level of skill in the art to perform amino acid substitutions using known protocols of recombinant gene technology including PCR, gene cloning, site-directed mutagenesis of cDNA, transfection of host cells, and in-vitro transcription. The variants can then be screened for functional activity. Most acceptable deletions, insertions and substitutions according to the present invention are those which do not produce radical changes in the characteristics of the protein in terms of its desired Met binding and its inhibition of Met activity. However, when it is difficult to predict the exact effect of the substitution, deletion or insertion in advance of doing so, one skilled in the art will appreciate that the effect can be evaluated by routine screening assays such as those described here, without requiring undue experimentation.

Derivatives of NK1

A “derivative” of any one of the novel NK1 polypeptides includes additional chemical moieties not normally a part of the protein. Covalent modifications of the protein are included within the scope of this invention. Such modifications may be introduced into the molecule by reacting targeted amino acid residues with an organic derivatizing agent that is capable of reacting with selected side chains or terminal residues. Such chemically modified and derivatized moieties may improve the protein's solubility, absorption, biological half life, binding affinity, and the like. These changes may eliminate or attenuate undesirable side effects of the protein in vivo. Moieties capable of mediating such effects are disclosed, for example, in Remington's Pharmaceutical Sciences, Mack Publishing Company, Easton Pa. (Gennaro 18th ed. 1990).

Cysteinyl residues most commonly are reacted with *alpha.-haloacetates (and corresponding amines) to give carboxymethyl or carboxyarmdomethyl derivatives. Cysteinyl residues also are derivatized by reaction with bromotrifluoroacetone, *alpha.-bromo-.beta.-(5-imidozoyl-)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate (pH 5.5-7.0) which agent is relatively specific for the histidyl side chain. p-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents reverses the charge of the lysinyl residues. Other suitable reagents for derivatizing .alpha.-amino-containing residues include imidoesters such as methylpicolinimidate; pyridoxal phosphate; pyridoxal; chloroborohydride; trinitrobenzenesulfonic acid; O-methylisourea, 2,4 pentanedione; and transaminase-catalyzed reaction with glyoxylate.

Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pK.sub.a of the guanidine functional group. Furthermore, these reagents may react with the groups of lysine as well as the arginine .epsilon.-amino group.

Modification of tyrosyl residues has permits introduction of spectral labels into a protein or peptide. This is accomplished by reaction with aromatic diazonium compounds or tetranitromethane. Most commonly, N-acetylimidizol and tetranitromethane are used to create O-acetyl tyrosyl species and 3-nitro derivatives, respectively.

Carboxyl side groups (aspartyl or glutamyl) are selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl)carbodiimide.

Aspartyl and glutamyl residues are converted to asparaginyl and glutaminyl residues by reaction with ammonium ions. Conversely, glutaminyl and asparaginyl residues may be deamidated to the corresponding glutamyl and aspartyl residues. Deamidation can be performed under mildly acidic conditions. Either form of these residues falls within the scope of this invention.

Derivatization with bifunctional agents is useful for cross-linking the polypeptide to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane.

Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other chemical modifications include hydroxylation of proline and lysine, phosphoiylation of the hydroxyl groups of seryl or threonyl residues, methylation of the .alpha.-amino groups of lysine, arginine and histidine side chains (T. E. Creighton, Proteins: Structure and Molecule Properties, W.H. Freeman & Co., San Francisco, pp. 79-86 (1983)), acetylation of the N-terminal amine, and, in some instances, amidation of the C-terminal carboxyl group.

Pharmaceutical Compositions and Kits:

In another embodiment, the present invention is a pharmaceutical composition of one of the disclosed NK1-based polypeptides, the NK1-based polypeptides with conservative amino acid substitutions, and derivatives thereof. In said composition, the polypeptide is dissolved in a pharmaceutically acceptable carrier or diluent, preferably an aqueous carrier. A variety of aqueous carriers or diluents can be used. These solutions are preferably sterile and generally free of undesirable matter. These compositions may be sterilized by conventional, well known sterilization techniques.

Pharmaceutically acceptable carriers can contain a physiologically acceptable compound that acts, e.g., to stabilize the composition or to increase or decrease the absorption of the agent and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the adjunctive and cancer agents, or excipients or other stabilizers and/or buffers. Detergents can also used to stabilize the composition or to increase or decrease the absorption of the pharmaceutical composition.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of a pharmaceutically acceptable carrier, including a physiologically acceptable compound depends, for example, on the route of administration of the adjunctive anti-cancer agent and on the particular physio-chemical characteristics of the adjunctive anti-cancer agent.

The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that polypeptides, when administered orally, may be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

Also within the scope described herein is a kit for providing a therapeutically effective amount of a bioactive agent for use in treating a patient, comprising a container, a volume of material stored within the container in substantially sterile form; wherein the material comprises the pharmaceutical of one of the present NK1-based polypeptides, the NK1-based polypeptides with conservative amino acid substitutions, or derivatives thereof. The kit can further contain at least one additional reagent.

Polynucleotides, Vectors, and Host Cells:

A polynucleotide of the present invention is one which encodes a polypeptide of the invention as defined above. This includes DNA and RNA polynucleotides. A polynucleotide of the invention may be single or double stranded. Generally, a polynucleotide according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression.

Sequences encoding all or part of the polypeptides of the invention and/or its regulatory elements can be readily prepared by the skilled person using the information and references contained herein and techniques known in the art (for example, see Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992). These techniques include the use of site directed mutagenesis of nucleic acid encoding NK1. A polypeptide according to the present invention may be isolated and/or purified (e.g. using an antibody) for instance after production by expression from encoding nucleic acid (for which see below). Polypeptides according to the present invention may also be generated wholly or partly by chemical synthesis, for example in a step-wise manner. The isolated and/or purified polypeptide may be used in formulation of a composition, which may include at least one additional component, for example a pharmaceutical composition including a pharmaceutically acceptable excipient, vehicle or carrier. A composition including a polypeptide according to the invention may be used in prophylactic and/or therapeutic treatment.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

Preferably, a polynucleotide of the invention in a vector is operably linked to a control sequence which is capable of providing for the expression of the coding sequence by the host cell, i.e. the vector is an expression vector.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage phagemid or baculoviral, cosmids, YACs, BACs, or PACs as appropriate. Vectors include gene therapy vectors, for example vectors based on adenovirus, adeno-associated virus, retrovirus (such as HIV or MLV) or alpha virus vectors.

The vectors may be provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in methods of gene therapy. Systems for cloning and expression of a polypeptide in a variety of different host cells are well known.

Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others.

Promoters and other expression regulation signals may be selected to be compatible with the host cell for which the expression vector is designed. For example, yeast promoters include S. cerevisiae GAL4 and ADH promoters, S. pombe nmt1 and adh promoter. Mammalian promoters include the metallothionein promoter which is can be included in response to heavy metals such as cadmium. Viral promoters such as the SV40 large T antigen promoter or adenovirus promoters may also be used. All these promoters are readily available in the art.

The vectors may include other sequences such as promoters or enhancers to drive the expression of the inserted nucleic acid, nucleic acid sequences so that the polypeptide is produced as a fusion and/or nucleic acid encoding secretion signals so that the polypeptide produced in the host cell is secreted from the cell.

Vectors for production of polypeptides of the invention of for use in gene therapy include vectors which carry a mini-gene sequence of the invention.

Vectors may be introduced into a suitable host cell as described above to provide for expression of a polypeptide of the invention. Thus, in a further aspect the invention provides a process for preparing polypeptides according to the invention which comprises cultivating a host cell carrying an expression vector as described above under conditions to provide for expression by the vector of a coding sequence encoding the polypeptides, and recovering the expressed polypeptides. Polypeptides may also be expressed in in-vitro systems, such as reticulocyte lysate.

A further embodiment of the invention provides host cells carrying the vectors for the replication and expression of polynucleotides of the invention. The cells will be chosen to be compatible with the said vector and may for example be bacterial, yeast, insect or mammalian.

The introduction of vectors into a host cell may be followed by causing or allowing expression from the nucleic acid, e.g. by culturing host cells (which may include cells actually transformed although more likely the cells will be descendants of the transformed cells) under conditions for expression of the gene, so that the encoded polypeptide is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers.

A further aspect of the present invention provides a host cell containing nucleic acid as disclosed herein. The polynucleotides and vectors of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

Methods of Treatment:

The polypeptides and polynucleotides of the present invention can be used in a method for treating Met-related conditions or diseases. Such treatment will be directed to treating a patient in need of controlling cell growth, cell proliferation, cell survival, or cell motility.

Polypeptides may be administered in any suitable form, for example in a pharmaceutical composition such as water, saline, dextrose, glycerol, ethanol and the like. Compositions may be formulated for injection, for example for direct injection to the site of intended treatment or intravenous injection.

Suitable doses or amounts of polypeptides will ultimately be at the discretion of the physician taking account of the nature of the condition to be treated and the condition of the patient. In general, dosage ranges will be 1 Âμg to 1 mg per kg body weight. The polypeptides may be administered by any suitable route, e.g. by i.v. or i.p injection, or directly to the site of treatment.

The polypeptides of the invention are useful for and can be delivered alone or as pharmaceutical compositions by any means known in the art, e.g., systemically, regionally, or, locally; by intraarterial, intratumoral, intravenous (IV), parenteral, intrapleural cavity, topical, oral, or local administration, as subcutaneous, intra-tracheal (e.g., by aerosol) or transmucosal (e.g., buccal, bladder, vaginal, uterine, rectal, nasal mucosa), intratumoral (e.g., transdermal application or local injection). Particularly preferred modes of administration include intraarterial injections, especially when it is desired to have a “regional effect,” e.g., to focus on a specific organ (e.g., brain, liver, spleen, lungs). For example, intra-hepatic artery injection is preferred if the anti-tumor regional effect is desired in the liver; or, intra-carotid artery injection, where it is desired to deliver a composition to the brain (e.g., for treatment of brain tumors), a carotid artery or an-artery of the carotid system of arteries (e.g., occipital artery, auricular artery, temporal artery, cerebral artery, maxillary artery, etc.).

Methods of Converting an Agonist to an Antagonist:

The ability to design specific mutations in the NK1 dimer interface that selectively disrupts NK1's ability to induce Met dimerization but retains its Met binding activity provides a mechanistic basis for designing Met antagonists for therapeutic applications. In order to serve as an RTK agonist, growth factors like HGF must have at least two functions, namely receptor binding and receptor activation. Thus, a mutated growth factor with selective disruption of its receptor activation ability but not its receptor binding may function as an RTK antagonist. Since activation of RTK is proposed to be mediated through a conserved mechanism of receptor dimerization or oligomerization, the disclosed method for design of NK1-based Met antagonists also provides a new concept for designing antagonists of other ligand-activated tyrosine kinase receptors. That is, the ability to separate Met binding of NK1 from its Met activation function has implications for antagonist design of other growth factor-activated tyrosine kinase receptors, i.e., by selectively abolishing the receptor activation ability but not the receptor binding of the growth factor. The methods disclosed herein can be used for designing an RTK antagonist (i.e., converting an RTK agonist to an RTK antagonist) for any RTK that is activated by receptor dimerization or oligomerization.

The family of ligand-activated receptor tyrosine kinases has a wide spread role in tumorigenesis and metastasis (Blume-Jensen and Hunter, 2001). The disclosed mechanistic-based design of Met antagonists also has important implications for other growth factor/tyrosine kinase receptor systems. RTKs active in such growth factor-RTK systems include epidermal growth factor receptor (EGFR), nerve growth factor receptor (NGFR), vascular endothelial growth factor receptor (VEGFR), fibroblast growth factor receptor (FGFR), stem cell factor receptor, and macrophage-stimulating protein receptor (RON).

Like Met, VEGF, NGF and EGF receptors are activated through growth factor-induced receptor dimerization. To activate their receptors, NGF and VEGF contain separate receptor binding and dimerization surfaces (Wiesmann et al., 1997; Wiesmann et al., 1999). Based on the work disclosed herein with NK1 and Met, one can design VEGF-based and NGF-based antagonists by separating their receptor binding activity from their receptor dimerization activity. In the case of EGF, ligand binding induces conformational changes of the receptor that mediates direct receptor/receptor dimerization (Cho and Leahy, 2002; Ferguson et al., 2003; Garrett et al., 2002; Ogiso et al., 2002). A mutated EGF may induce a distinct conformational change of the receptor that is incompatible with receptor dimerization and activation. Given the relative small size of VEGF and EGF, such growth factor-based antagonists provide an exciting alternative to the current antibody-based approach.

Thus, the present invention also includes a method for designing an antagonist by converting a growth factor from a receptor tyrosine kinase agonist to an antagonist, wherein the native receptor dimerizes or oligomerizes to become active. The experimental design and methods utilized and set forth in the Examples below provide one means for performing such method. Steps for carrying out this method include: (1) identifying the region of the growth factor that promotes dimerization or oligomerization of the receptor (e.g., through crystallographic analysis); (2) providing mutants wherein at least one amino acid in the region of the growth factor that promotes said dimerization or oligomerization has been substituted with another amino acid; (3) screening these mutants to determine receptor dimerization or oligomerization activity and to determine the binding affinity of the mutants to the receptor; and (4) selecting the mutants that have a decreased ability to promote dimerization or oligomerization of the receptor but retain the ability to bind the receptor. More specifically, as described in the Examples, a proximity assay such as an AlphaScreen assay (Perkin Elmer, Waltham, Mass.), may be used to screen for and determine receptor dimerization or oligomerization activity or to determine the binding affinity of said mutants to the receptor. Further, as shown in the Examples, the binding affinity of the mutants to the receptor can be assayed through competition experiments with the wild type ligand.

Having now generally described the invention, the same will be more readily understood through reference to the following examples, which are provided by way of illustration, and are not intended to be limiting of the present invention, unless specified.

### EXAMPLES

### Example 1

### Experimental Procedures for Examples 2-6

Production of NK1 Proteins

The human HGF NK1 (residues 28-209) was expressed as a 6×His-thioredoxin fusion protein from the expression vector pET-Duet1 (Novagen). The fusion protein contains a His6-Tag (MEHHHHHHMS) at the N terminus and a thrombin protease site between thioredoxin and NK1. Protein was expressed in the E. coli strain Rosetta/gami(DE) (Novagen) to promote disulfide bond formation. Bacterial cells transformed with the expression plasmid were grown in LB broth to an OD550 of ˜0.5 to 1.0 and induced with 100 μM isopropyl β-D-1-thiogalactopyranoside (IPTG) and 0.1% lactose for overnight at 22° C. Cells were harvested, resuspended in 200 ml extract buffer (495 mM NaCl, 25 mM Tris-HCl, pH 8.0, 5 mM imidazole, and 1 mg/ml lysozyme) per 12 liters of cells, and sonicated. The lysate was centrifuged at 20,000 rpm for 30 min, and the supernatant was loaded on to a 50 ml Ni-NTA agarose column. The column was washed with 500 ml extract buffer and eluted with buffer containing 25 mM Tris-HCl, pH8.0, and 500 mM imidazole. The eluted protein was cleaved overnight with thrombin at a protease/protein ratio of 1:1000 in the cold room and applied to a 5 ml HiTrap heparin HP column (Amersham Biosciences). The column was eluted with a gradient of 0.1 to 1.2 M NaCl, 25 mM Tris-HCl pH 8.0, where the 6×His-thioredoxin tag was in the flow through and NK1 was eluted at 800-1000 mM NaCl. Protein used for crystallization was further purified with gel filtration and cat ion exchange columns. Mouse NK1 and human NK1 mutants were purified with the same protocols.

Crystallization, Data Collection, and Structure Determination

For crystallization, proteins were concentrated to approximately 10 mg/ml in 25 mM Tris-HCl pH 8.0 and 100 mM NaCl. Crystals were grown in hanging drops containing equal volumes of protein and well solution. Crystallization conditions for human HGF/NK1 were 50 mM ammonium sulfate, 26-29% PEG 4000 (w/w), 100 mM Tris-HCl pH 8.0, 0.5 mM 0-octyl glucoside (βOG), and 5% ethylene glycol while crystallization conditions for mouse NK1 were 50 mM ammonium sulfate, 26-32% PEG 1000, 50 mM Tris-HCl pH 8.0, and 5% ethylene glycol. Crystals grew over weeks to months to a size of several hundred microns.

Data were collected with a MAR 225 CCD detector at 5-ID (DND-CAT) of the Advance Photon Source (APS) located at Argonne National Laboratory. Crystals were flash frozen in liquid nitrogen after a quick wash in cryoprotectant solution containing 50 mM ammonium sulfate, 29% PEG 4000 or 1000 (w/w) (human or mouse NK1 respectively), 100 mM Tris-HCl pH 8.0, 0.5 or 0 mM βOG, 100 mM NaCl, and 15% ethylene glycol. Data was integrated and scaled with HKL2000 (Otwinowski and Minor, 1997). The human NK1 crystallized in space group P21, a=49.40 Å, b=51.81 Å, c=73.17 Å, and β=107.87°, and the mouse NK1 in P63 with a=b=86.49 Å, c=104.45 Å. The structure was solved by molecular replacement using the PDB coordinates 1NK1. Molecular replacement and model refinement were performed with CNS, where twin fraction was incorporated for the refinement for the mouse structure (Brunger et al., 1998). Manual model building was done with the program 0 (Jones et al., 1991).

Production of Biotinylated NK1

The biotinylated NK1 was produced by fusing the 20 amino acid biotin acceptor peptide sequence from the pDW464 plasmid (Duffy et al., 1998) to the N-terminus of NK1 in the pET-Duet1 vector containing the His6-thioredoxin-NK1 fusion protein. This plasmid was coexpressed with the E. coli biotin holoenzyme synthetase (BirA) from a pACYC-Duet1 vector (Novagen). The biotinylated NK1 was purified and digested with thrombin as for native NK1, and was further purified with a monomeric avidin column (Pierce).

Production of the Met Extracellular Domain

The Met protein (residues 25-567, containing the sema domain and the cysteine-rich domain) was expressed as a C-terminal hexahistidine tag fusion protein from Lec 3.2.8.1 cells (Gherardi et al., 2003). Cells were grown in a 1:1 mixture of alpha-MEM and DMEM media with 2.5% FBS. Media was harvested every 5 days and concentrated prior to loading on a 50 ml nickel column (Qiagen) equilibrated with 25 mM Tris-HCl pH 7.5, 25 mM imidazole, 150 mM NaCl, and 10% glycerol. The column was washed with equilibrated buffer above until the absorbance at 280 nm reached baseline and then the protein was eluted with 175 mM imidazole, 25 mM Tris-HCl pH 7.5, and 10% glycerol. The eluted protein was dialyzed with 50 mM citrate pH 6.0, 100 mM NaCl, and 10% glycerol, and treated with endoglycosidase Hf (2.5 units per 1 μg of Met) were added at room temperature for 24 hours. Deglycosylated Met was loaded onto a 5 ml HiTrap blue HP column (Amersham Biosciences) and the column was washed with 5 to 10 column volumes of 20 mM Tris-HCl pH 8.0 and 200 mM NaCl. Met was eluted with 20 mM Tris-HCl pH 8.0 and 800 mM NaCl. Prior to use in AlphaScreen assays the protein was concentrated and the buffer exchanged with 25 mM Tris-HCl pH 8.0 and 100 mM NaCl in Amicon ultra centrifugal concentrators.

AlphaScreen Assays for NK1-Met Binding and Met Dimerization

The binding of NK1 to Met was determined by AlphaScreen assays using a hexahistidine detection kit from Perkin-Elmer. The experiment was performed using 10 nM biotin-NK1 and 6his-Met in the presence of 5 μg/ml donor and acceptor beads in a buffer containing 50 mM MOPS, pH7.4, 50 mM NaF, 0.05 mM CHAPS, and 0.1 mg/ml bovine serum albumin. Competition experiments were performed with 0.01 to 2.5 μM untagged ligand in the presence of 1 μM heparin and IC₅₀ values for NK1-Met interaction were determined from a nonlinear least square fit of the data. All experiments were duplicated or triplicated with standard errors typically less than 10% of the measurements. The Met dimerization assays were prepared using 40 nM of 6his-Met in the presence of 5 μg/ml nickel chelate donor and acceptor beads (Perkin-Elmer). The effect of Met ligands and heparin on Met dimerization was determined using 1 μM untagged ligand and 1 μM heparin.

MDCK Cell Scatter Assay

HGF, wild type and mutated NK1, and heparin were used in the assay at final concentrations 60 ng/mL, 1 M, and 2 μM, respectively. The NK1 wt and mutants, heparin, or DMEM containing 5% FBS, 1% penicillin/streptomycin, and 1% L-glutamine were added to the appropriate wells of a 96-well plate. MDCK cells were diluted in the above media to 0.83×105 cell/mL and seeded into the 96-well plate at 0.083×105 cell/well. The cells were mixed by gently rocking the plate and incubated 10 min at 37° C., 5% CO2. After the incubation, HGF was added to the appropriate wells of the plate and incubated at 37° C., 5% CO2 overnight. The cells were fixed and stained using 0.5% crystal violet in 50% ethanol at room temperature for 10 min, washed with water, and allowed to dry prior to taking photos.

uPA-Plasmingen Assay

MDCK cells were seeded in a 96-well plate at 1500 cells per well and incubated overnight at 37° C., 5% CO₂ in DMEM containing 10% FBS and 1% penicillin/streptomycin. HGF, NK1 wt and mutant, and heparin were used in the assay at final concentrations of 60 ng/mL, 1 μM, and 2 μM, respectively. NK1 wt or mutant and heparin were added to the cells and incubated 15 min at 37° C., 5% CO2. HGF was then added to the appropriate wells and incubated 24 hr at 37° C., 5% CO₂. The plates were processed by first washing wells twice with phenol-red free DMEM, then adding 200 uL of reaction buffer, containing 50% (v/v) 0.05 units/mL plasminogen (Roche) in phenol-red free DMEM, 40% (v/v) 50 mM Tris buffer (pH 8.2), and 10% (v/v) 3 mM Chromozyme PL (Roche) in 100 mM glycine solution, to each well. The plates were incubated 4 hr at 37° C., 5% CO₂, after which time the absorbance of each well was read using an automated spectrophotometric plate reader at 405 nm.

Thymidine Incorporation Assays

The highly proliferative sub-clone of MDCK cells were distributed into 96-well plates at 2×10³ cells per well with DMEM supplemented with 10% FBS for 24 h. The cells were starved in DMEM without FBS for 24 h and treated with Heparin alone or Heparin in combination with wild type NK1 or various NK1 mutants for 1 h. Then the cells were further incubated with or without HGF/SF for 12 h. One microcurie of [3H]thymidine (Amersham) was added 4 h before analysis. Cells were washed with PBS and [3H]thymidine incorporation was measured by precipitation of whole cells with chilled 10% trichloroacetic acid, solubilization of precipitates with lysis buffer (0.02 M NaOH/0.1% SDS), suspension in 150 μl of scintillation mixture (Packard Bioscience) and measurement by a liquid scintillation counter (MicroBeta TriLux, PerkinElmer).

Branching Morphogenesis Assays

Branching morphogenesis in 3D Matrigel matrix was analyzed as follows: cells at a density of 50,000 cells/ml in DMEM supplemented with 10% FBS medium were mixed with an equal volume of Matrigel (Becton Dickinson), plated at 100 μl per well on a 96-well culture plate, and incubated for 30 min at 37° C. with 5% CO₂ to allow gel formation. Cells were treated with Heparin alone or Heparin in combination with wild type NK1 or various NK1 mutants for 1 h. Growth medium with or without 100 ng/ml of human HGF/SF was introduced into each well. The cells were photographed at different time points.

### Example 2

### Direct Binding of the Human and Mouse NK1 to the Met Extracellular Domain

To understand the detailed biochemical mechanisms of Met binding and activation by NK1, mouse and human NK1, the human NK1 mutant R134G, and the first 567 residues of the Met extracellular domain, which contains the sema and cystine-rich domains, were expressed and purified (FIG. 1A and Example 1). The purified Met extracellular domain (residues 25-567 with a C-terminal histidine tag) consists of a 35 KD α-chain (residues 25-307) and a 32 KD β-chain (residues 308-567) (Gherardi et al., 2003). Both mouse and human NK1 run at a 21 KD band but the human NK1 expressed in E. coli was consistently contaminated with an N-domain truncation product (lane 2, FIG. 1B) due to the presence of arginine 134 (R134) in the human NK1 that is sensitive to cleavage by proteases (Pediaditakis et al., 2002). Replacement of R134 with a glycine, the residue present in mouse NK1, removed the protease site (lane 3, FIG. 1B) and improved the yield of the full length NK1 product. A biotinylated version of the human NK1 was also expressed in bacteria and purified for Met binding assays (lane 4, FIG. 1B).

To determine the functional activity of the above purified proteins, the direct interactions of Met with the biotinylated NK1 were measured using AlphaScreen assays as illustrated in FIG. 1C. In this assay, streptavidin donor beads and nickel chelated acceptor beads were attached to biotinylated NK1 and histidine-tagged Met, respectively. When NK1 interacts with Met, excitation of a laser beam at 680 nm causes the donor beads to emit single oxygen molecules to activate the fluorophores in the acceptor beads and a light signal is detected at 520-620 nm. As shown in FIG. 1D, incubation of NK1 with Met plus heparin yielded ˜80,000 photon counts versus <300 photon counts produced by either NK1 or Met alone. Importantly, interaction of NK1 with Met was dependent on the presence of heparin. Addition of heparin increased the NK1-Met binding signal by >200 fold, consistent with the fact that Met activation by NK1 requires the presence of heparin as a Met co-receptor (Catlow et al., 2003; Lyon et al., 2004; Rubin et al., 2001; Schwall et al., 1996).

To determine the binding affinities of the various NK1s for Met, competition experiments were performed using unlabeled NK1 proteins (FIG. 1E). Both mouse NK1 and human R134G mutant bind to Met with a similar affinity (IC₅₀ of ˜30 nM), where as the human NK1 binds to Met with a 3-fold weaker affinity (IC₅₀ of ˜100 nM). We also measured the binding affinity of the full length HGF, which binds to Met with an IC₅₀ of 0.5-5.0 nM (data not shown), which is close to the range of HGF potency as measured in cell based assays (Gao et al., 2005; Xie et al., 2005). These quantitative measurements thus establish that the affinities of the NK1 fragments are 10-50 folds weaker than the full-length HGF.

### Example 3

### Met Dimerization is Promoted by NK1 Binding

To probe the mechanism of Met activation by NK1, we designed a Met dimerization assay based on AlphaScreen technology (FIG. 2A). In this assay, both nickel chelate donor and acceptor beads were attached to Met via its C-terminal histidine tag. When Met formed an NK1-mediated dimer, the dimerization signal was recorded. As shown in FIG. 2B, the Met was monomeric regardless of the presence of heparin (<500 photo counts, FIG. 2B). Addition of human NK1 and heparin dramatically increased the Met dimerization signal to >40,000 photo counts. In the absence of heparin, NK1 only promotes a basal Met dimerization signal. Similar results were obtained with the mouse NK1 and the human R134G NK1. Furthermore, in a dose titration experiment (FIG. 2C), we found that NK1 induced Met dimerization with an effective concentration (EC50) ranging from 50 to 200 nM, which is closely related to the binding affinities of NK1 for Met from FIG. 1E. Together, these results suggest that NK1 is capable of binding and inducing Met dimerization in a heparin dependent manner.

The AlphaScreen is a proximity assay which can generate signals arising from Met dimerization or oligomerization. To determine the nature of the Met dimer/oligomer, we performed dynamic light scattering analysis to determine the hydraulic diameter of the Met/NK1 complex. In this assay, we used NK1 as our positive control since it has been shown to form a dimer in a heparin dependent manner (Chirgadze et al, 1998). In the absence of heparin, dynamic light scattering revealed that NK1 forms a mono-dispersed monomer with a hydraulic diameter of 54 Å (FIG. 2D). The referred molecular weight from this measurement for the NK1 monomer is 33 KD, which is overestimated from the real molecular weight of NK1 at 21 KD due to the elongated shape of NK1 monomer structure (see FIG. 3B). Addition of heparin increased the hydraulic diameter of NK1 to 66 Å with a referred molecular weight of 51 KD, which is better correlated with the expected molecular weight of 48 KD for a 2:2 NK1 dimer/heparin complex, where heparin used has an average molecular weight of 3 KD. Regardless of the presence of heparin, the Met sema domain is monomeric with a mono-dispersed diameter of 82-84 Å and an estimate molecular weight of 95 KD, which is also slightly overestimated from its expected molecular weight of 63 KD because of the elliptical structure of the Met sema domain (Stamos et al, 2004). Addition of NK1 plus heparin to Met resulted in a mono dispersed complex (SOS<1.0) with its hydraulic diameter increased to 114 Å. The estimated molecular weight from this measurement for this complex is 194 KD, which is in close agreement with the expected molecular weight of 174 KD for a 2:2:2 Met/NK1/heparin complex, suggesting that in the presence of heparin Met and NK1 form a uniform dimer rather than a higher oligomeric complex. In the absence of heparin, NK1 and Met also form a dimeric complex which, however, is poly dispersed and highly unstable as indicated by the high SOS value (FIG. 2D). The Met sema domain (residues 25-567) used in our experiments has been shown to be necessary for Met dimerization in cell based cross-linking experiments (Kong-Beltran et al., 2004). The results here further showed that the Met sema domain is sufficient to form a dimeric complex with NK1 in vitro.

### Example 4

### Crystal Structures of the Mouse and Human NK1

To investigate the molecular mechanism by which NK1 induces Met dimerization, the crystal structure of mouse NK1 at a 2.4 Å resolution and a high resolution structure of human NK1 (1.8 Å) were determined for structural comparison. The statistics for the crystallographic data and refined structures are summarized in Table 3.

TABLE 3
Statistics of Crystallographic Data and Structures.

| | Human NK1 | | Mouse NK1 | |
|---|---|---|---|---|
| Data collection | | | | |
| APS Beam Line | 5-ID | | 5-ID | |
| Space Group | P2₁ | | P6₃* | |
| Resolution (Å) | 50.0-1.80 | (1.86-1.80) | 50.0-2.37 | (2.45-2.37) |
| Unit cell | a = 49.40 Å, b = 51.81 | | a = b = 86.49 Å, | |
| parameters | Å, c = 73.17 Å, | | c = 104.45 Å | |
| | β = 107.87° | | | |
| Unique reflections | 28,061 | (1,004) | 13,513 | (225) |
| Completeness (%) | 86.8 | (31.3) | 75.6 | (12.6) |
| I/σ | 12.9 | (1.3) | 23.2 | (2.2) |
| Redundancy | 3.7 | (1.7) | 8.7 | (1.5) |
| R_{sym} | 0.080 | (0.37) | 0.079 | (0.20) |
| Refinement | | | | |
| Resolution (Å) | 50.0-1.80 | (1.86-1.80) | 50.0-2.5 | (2.61-2.5) |
| # of reflections | 26,701 | (666) | 23,321 | (580) |
| # of residues | 348 | | 344 | |
| # of solvent mol. | 330 | | 85 | |
| # of sulfate ions | 4 | | 3 | |
| # of non-H atoms | 3165 | | 2878 | |
| R_{cryst} | 0.201 | (0.324) | 0.173 | (0.285) |
| R_{free} | 0.248 | (0.396) | 0.231 | (0.313) |

| | | |
|---|---|---|
| Rmsd bonds (Å) | 0.006 | 0.008 |
| Rmsd angles (°) | 1.25 | 1.43 |
| Average Bfactor | 22.75 | 41.82 |
| (Å²) | | |

*twin frac. = 0.433

FIG. 3A shows the overall structure of mouse NK1, which forms a disc-shape head-to-tail dimer with approximate overall dimensions of 60×56×33 Å. Each monomer in the dimer comprises two globular domains: the N-terminal heparin binding domain and the C-terminal kringle domain, connected by a two-residue linker (R126 and N127). The shape of the NK1 monomer is relatively elongated with an approximate size of 50×27×22 Å (FIG. 3B). The maximum dimensions of the NK1 dimer and monomer structures are remarkably correlated with the hydraulic diameter of the NK1 dimer and monomer measured by dynamic light scattering (FIG. 2D).

The head-to-tail NK1 dimer is formed by the intertwined packing between the N-domain and the C-terminal kringle domain of the two monomers that bury 2200 A2 of solvent accessible area. The interactions between the two monomers are summarized in FIG. 3B. The core hydrophobic interface comprises reciprocal interactions of Y124 with V140 and P204 (FIG. 3C), as well as the packing of the C128-C206 disulfide bond from both monomers (FIG. 3B). The extensive NK1 dimer interface includes two pairs of charged interactions of K85-D202 and K122-D123 (FIGS. 3C&D) and a network of hydrogen bonds mediated by N127 (FIG. 3D). N127 from both monomers is located in the center of the dimer interface with its side chain packing with each other. In addition to these packing interactions, the side chain of N127 of the one monomer mediates reciprocal interactions by forming three pairs of hydrogen bonds with main chain amide of N127 and the two carbonyl oxygens of D123 and I125 from the other monomer (FIG. 3D). Together, these hydrophilic interactions are the key determinants of the overall specificity and stability of the NK1 dimer complex

The mouse NK1 is 90.0% identical in sequence to the human NK1 (FIG. 3F; SEQ ID NO: 2 and 1, respectively) and its monomer structure and dimer arrangement resemble those of the human NK1 structure reported here (FIG. 3E) and of previously reported ones (Chirgadze et al., 1998; Lietha et al., 2001; Ultsch et al., 1998). The average root mean square deviation (rmsd) value between NK1 monomers is 1.12 Å for main chain atoms for all known structures. Much of this difference between monomer structures is due to the flexibility of the linker region as the core N-terminal and kringle domains vary even less. The average main chain atom rmsd for the N-terminal and kringle domain is 0.72 Å and 0.67 Å, respectively, for all determined NK1 structures. Notably, despite different buffer conditions and species of proteins used in crystallization, all NK1 structures determined to date contain a highly similar head-to tail dimer arrangement. Further, all residues involved in the formation of the NK1 dimer interface are conserved in species from chicken to human (FIG. 3F), suggesting that the observed NK1 dimer may perform an evolutionarily conserved function in HGF-Met signaling.

### Example 5

### Effects of Mutations in the NK1 Dimer Interface

To determine the role of the NK1 dimer configuration in the binding and activation of Met, four key residues (Y124, K85, D123, and N127) that form the dimer interface were mutated. These mutations were made in the human R134G background to facilitate purification. Since the dimer interface is far away from the heparin binding site located in the N domain (FIG. 3E), these dimerization mutants retained full heparin binding activity and were purified by a heparin affinity column to homogeneity for the Met dimerization and binding assays. As expected, NK1 promoted Met dimerization in a heparin dependent manner (FIG. 4A). All other four NK1 mutants dramatically reduced the ability to induce Met dimerization regardless of the presence of heparin. Specifically, three mutants, Y124A, K85A/D123A, and K85A/N127A, totally abolished Met dimerization activity (SEQ ID NO: 8, 10, and 11, respectively).

Also, the Met binding activity of these NK1 mutants was measured by the same competition experiments as first shown in FIG. 1D. In contrast to the Met dimerization activity, the Met binding activity of these NK1 mutants was little affected. As shown in FIG. 4B, the binding affinity for Met by two NK1 mutants is even slightly better than that for the wild type NK1 (IC50 values of 39 and 32 nM for the Y124A and K85A NK1s vs. 49 nM for NK1). These data suggest that the Met binding activity of NK1 can be completely separated from its ability to induce Met dimerization. Furthermore, the above NK1 dimerization mutants can dominantly inhibit Met dimerization induced by wild type NK1 (FIG. 4C), implying that they may potentially function as Met antagonists.

### Example 6

### NK1 Dimerization Mutants are Met Antagonists

To determine whether the above NK1 dimerization mutants can function as Met antagonists, we first performed protease assays for the urokinase-type plasminogen activator (uPA) since it is induced by HGF-mediated Met activation in MDCK cells and various cancer cell lines (Xie et al., 2005). In MDCK cells, uPA activity is consistently elevated by 4-5 folds by treatment with HGF (FIG. 5A). Treatment with the mouse and human NK1 or the human R134G NK1 mutant also induced uPA activity by 3-4 folds, while addition of the four dimerization mutants showed little effects (FIG. 5A). Furthermore, while wild type NK1 showed no inhibitory effects on HGF-mediated uPA activation, the four NK1 dimerization mutants significantly blocked HGF-induced uPA activity, demonstrating that they can function as dominant Met antagonist in this assay. Remarkably, the agonist and antagonist properties of these NK1 mutants are closely correlated with their ability to induce Met dimerization in AlphaScreen assays as shown in FIG. 4A.

Since Met activation by HGF also induces cell proliferation, we used thymidine incorporation to measure HGF-stimulated DNA synthesis. As shown in FIG. 5B, both HGF and wild type NK1 stimulated thymidine incorporation by 2-4 folds. The four NK1 mutants on their own had little effect but efficiently blocked HGF-mediated DNA synthesis, again consistent with the antagonist properties of these NK1 mutants.

HGF-mediated Met activation also affects cell morphological behavior e.g. cell scattering, branching, and invasion, thus we tested whether the NK1 mutants can inhibit the above HGF-mediated effects. As shown in FIG. 5C, the untreated MDCK cells grow as tightly packed colonies. Addition of HGF promotes dispersion of these MDCK colonies. While addition of the Y124A mutant did not disperse the cells, it dominantly inhibited HGF-induced cell scattering. HGF also induces branching morphogenesis of DU145, a prostate cancer cell line (FIG. 5D), or cell invasion of DBTRG, a glioblastoma cell line (FIG. 5E). The Y124A mutant can effectively inhibit both HGF-mediated activities, further corroborating its antagonist properties. The activities of these NK1-based Met antagonists on DU145 and DBTRG imply that they might have potential applications in cancer therapy.

### Example 7

### NK1 Mutants as Met Antagonists

Materials and Methods.

Mutagenesis and Cloning—Mutagenesis of the full length and NK 1 fragments was performed in a site directed manner, using PCR-based techniques. Selection of mutant clones from unmutated parental DNA strands was achieved via methylation sensitive restriction digestion with DpnI. Mutants were confirmed by DNA sequencing before being sub-cloned into either pA71d or pPIC9K (Invitrogen) for expression in mammalian cells or in the yeast P. pastoris, respectively.

Transfections—Wild type and mutant full length HGF/SF proteins were expressed transiently in the mouse neuroblastoma Neuro2A cells using Lipofectamine 2000 (Invitrogen). Concentration of expressed proteins within the supernatants was measured using a sandwich enzyme-linked immunoassay (R&D Systems, Cat # DY294). Stable transfectants in the mouse NS0 myeloma cell line were selected using Hygromycin B at 0.75 mg/ml. Clones expressing the protein of interest were identified by a slot blot assay using a polyclonal sheep anti-human HGF/SF antibody (1W53) and expanded for protein production.

Protein purification—Expression and purification of the NK1 mutants were carried out as described by others (Chiragiadze, D Y, Nat Struct, Biol. 6, 72-9 (1999); Lietha, D, Embo J., 20, 5543-55 (2001). Purification of the wild type and mutant full length HGF/SF proteins was carried from the supernatants of the relevant transfectants using Heparin-Sepharose (Cat # 17-0998-01; Amersham Biosciences) followed by Mono-S (Cat # 17-0547-01; Amersham Biosciences) chromatography. Proteins were stored in 0.05M MES, 1 M NaCl pH 6.0.

Surface plasmon resonance—SPR was carried out as described in Holmes (J. Mol. Bio. 367, 395-408 (2007)) except that PBS-EP rather than HBS-EP was used as the running buffer and 5 M NaCl was used for chip regeneration. Briefly, a CM5 chip was coated with MET567 by amine coupling chemistry. Ligand was then flowed through the cell at various concentrations to allow calculation of the dissociation equilibrium (K_{D}).

Small-angle X-ray scattering (SAXS) data collection and processing—Synchrotron X-ray scattering data were collected at the EMBL X33 beamline (DESY, Hamburg) 28 using a MAR345 image plate detector. The scattering patterns of all samples were measured at several solute concentrations ranging from 0.5 to 7.0 mg/ml. At a sample-detector distance of 2.7 m, the range of momentum transfer 0.1<<5 nm-1 was covered (s=4π sin(θ)/λ, where 2θ is the scattering angle and λ=0.15 nm is the X-ray wavelength). The primary data processing and evaluation of the overall structural parameters were performed using standard procedures by the program package PRIMUS 29. The molecular masses (MM) of the solutes were evaluated by scaling against reference solutions of bovine serum albumin. Particle shapes at low resolution were reconstructed by a multiphase bead modeling program MONSA (Svergun, Biophys J, 76, 2879-86 (1999). Two distinct phases were used to highlight the individual components (NK1 and MET567) in the complex. The modeling of the monomeric complex was constructed by simultaneous fitting of three scattering curves (free MET567, free LC and the 1:1 complex). In the case of the dimeric NK1-MET567 complex, the scattering curves from wild type NK1 and the complex were fitted and the modeling was constrained by P2 symmetry. Multiple MONSA runs yielded superimposable solutions and typical models are presented below.

Met Binding Assay—This was carried out using a competition assay using NUNC F96 Maxisorp 96-well plates (Cat # 442404) coated overnight with 50 ml/well of a 100 nM of a construct corresponding to the full length ectodomain (MET928) 21. Wells were blocked using a Reagent diluent (R&D Systems, Cat # DY995) before being incubated with mixtures of HGF/SF mutant proteins and 1×10-10M biotinylated wild type full length HGF/SF. Bound, biotinylated HGF/SF was detected using horseradish peroxidase-conjugated Streptavidin (DAKO Cat # P0397).

Western Blots—Western blots of ERK activation were performed as in Holmes (2007). Briefly, MK cells were treated with ligand for 5 mins, lysed and the level of ERK activation was measured by probing blots with antibodies against total and phosphorylated ERK1/2.

Cell motility assays—Madin-Darby canine kidney (MDCK) cells were used to seed 6-well tissue culture plates (Falcon) at a density of 2,500 cells per well. Cells were then incubated in DMEM (Gibco)+5% FCS (Gibco) at 37° C./5% CO₂ for 48 hours and next treated with test proteins in DMEM+5% FCS and incubated overnight. Photographs were taken using a Hamamatsu camera with a phase contrast lens at 100 times magnification.

DNA synthesis assay—DNA synthesis assays were carried out as in Holmes (2007). Briefly, mouse keratinocyte (MK) cells were grown to confluence and then treated with the various ligands in the presence of ³[H]-methylthymidine. Cells were then lysed and mixed with scintillant to measure levels of incorporation of the radoactive isotype into DNA.

Results.

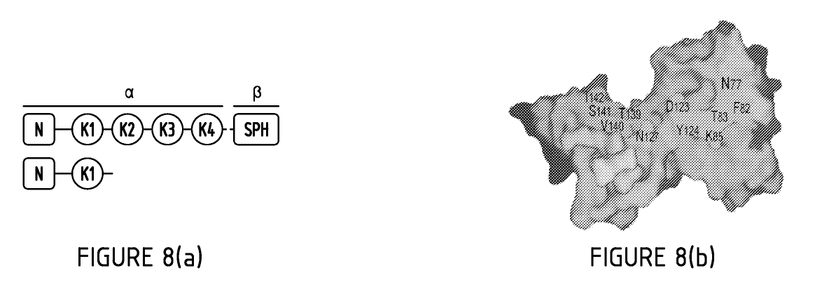

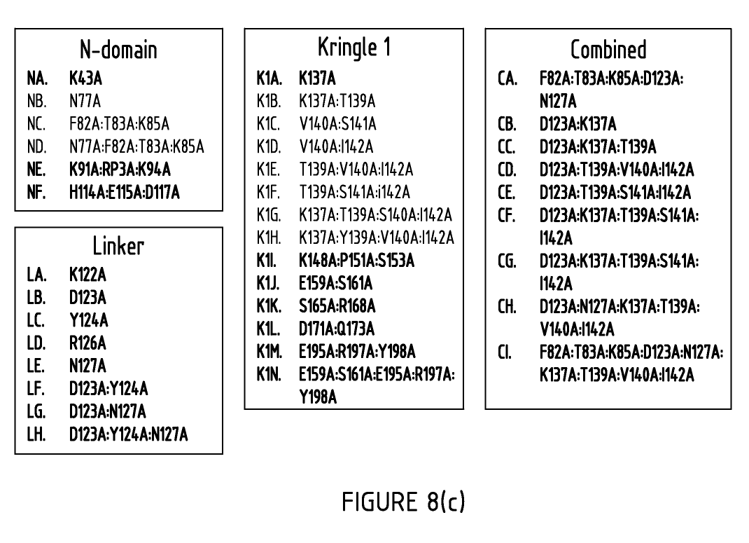

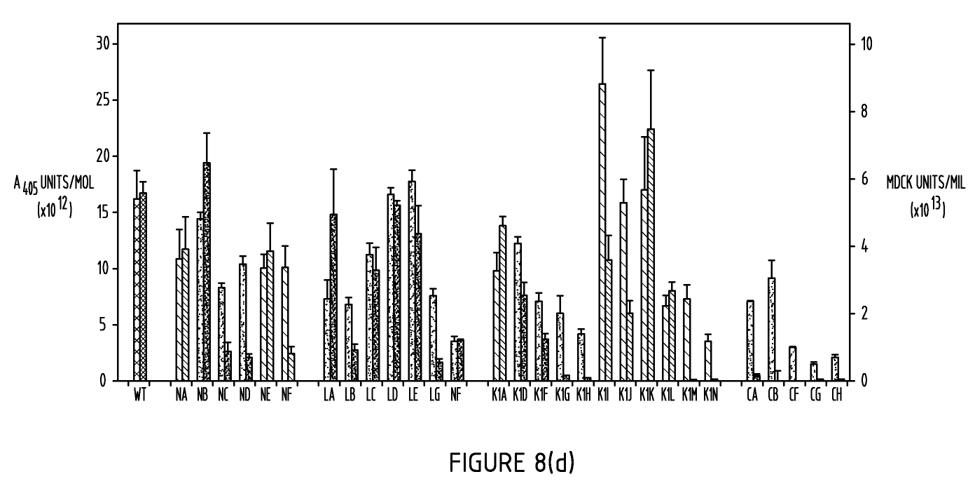

Several crystal structures of NK1 have consistently yielded a dimer arranged in a head to tail orientation (Chirgadze, D. Y. Nat Struct Biol, 6, 72-9 (1999); Lietha, D., Embo J., 20, 5543-55 (2001); Ultsch, M., Structure, 6, 1383-93 (1998); Watanabe, K, J. Mol. Bio, 319, 283-88 (2002). The dimer interface buries in excess of 2,000 A2 and consists of a central area in which the two inter-domain linkers (K122, D123, Y124, I125, R126 and N127) make extensive contacts and two adjacent areas in which residues between the N and K1 domains of different protomers make a number of further contacts. FIG. 8B shows, in yellow, selected residues at the NK1 dimer interface accounting >50% of the buried surface of each protomer.

An extensive number of individual or cluster alanine mutations have been introduced in the N and K1 domains and the inter-domain linker (FIG. 8C). These include mutations at the dimer interface (shown in yellow in FIGS. 1C and 1D) as well as mutations in other regions (shown in blue in FIGS. 1C and 1D). The latter include mutations at residues responsible for intra-protomeric contacts between the N and K1 domains (mutants NA, K1A, and K1L) (Chirgadze, D. Y. Nat Struct Biol, 6, 72-9 (1999)) as well as mutations at residues involved in MET binding (mutants NE, NF, K1J, K1M and K1N) (Hartmann, G [published erratum appears in Curr Biol 1998 Oct. 8; 8(20):R739], Curr Biol, 8, 125-34 (1998); Lokker, N. A., Protein Eng, 7, 895-903 (1994)) and located on the face of NK1 opposite to that involved in dimer assembly. The mutations were expressed in the background of both full length HGF/SF and NK 1. This is important as at least another HGF/SF domain, the C-terminal serine proteinase homology (SPH) domain (FIG. 8A) binds MET and is critical for receptor activation (Kirchhofer, D. et al., J. Bio Chem., 279, 39915-24 (2004)) and as a result the net effect of mutations in the N and K1 domains may differ in full length HGF/SF and NK 1.

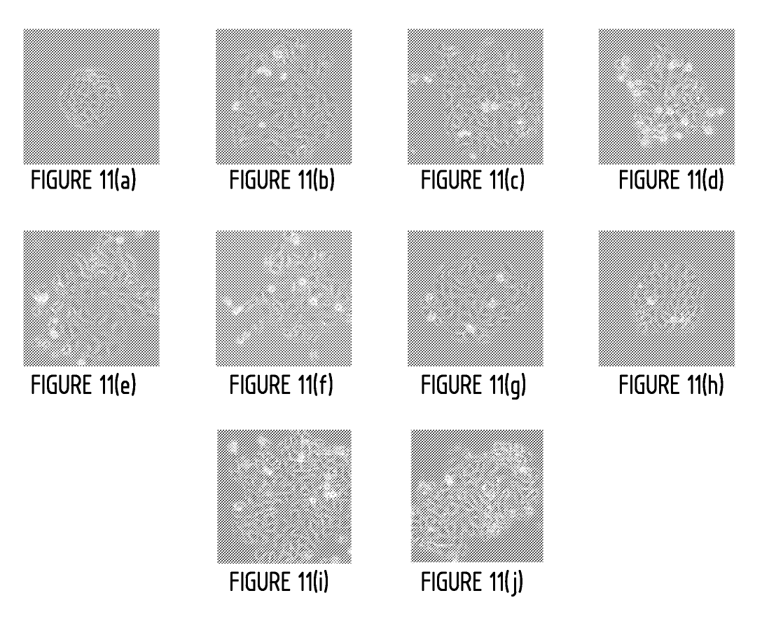

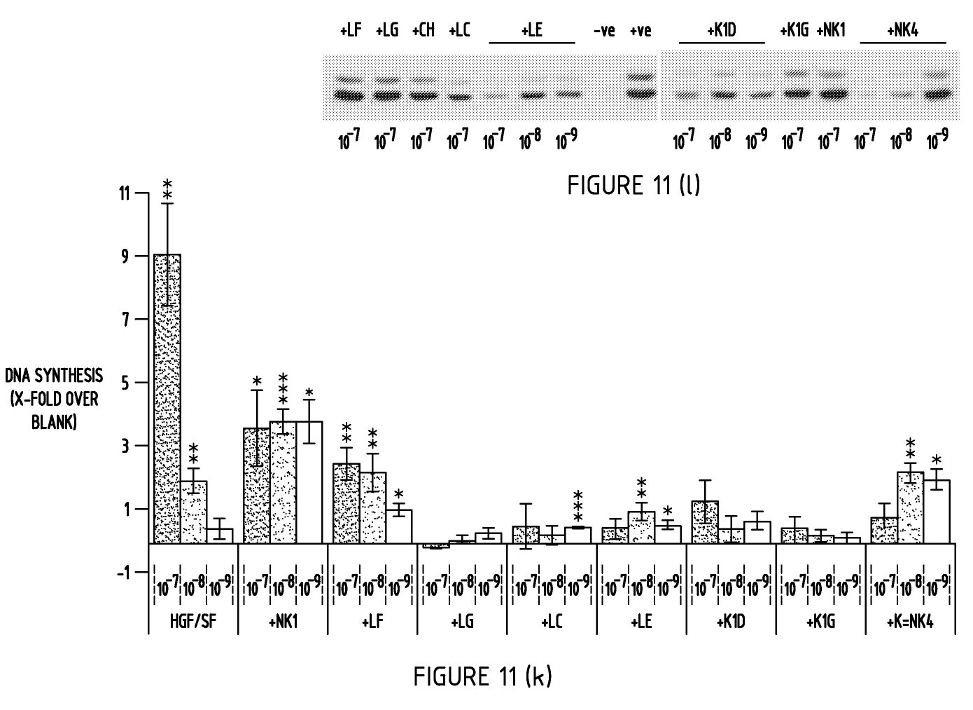

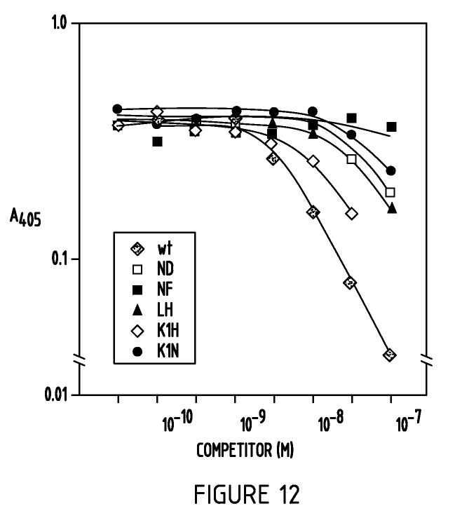

Wild type and mutant full length HGF/SF proteins were produced initially in Neuro2a cells by transient transfection and characterised for expression level, MET binding and MET activation. Eight mutants were not expressed well and were not characterised extensively. Data from the remaining 29 mutants are shown in FIG. 8D and, for each mutant, two columns are shown: the one on the left shows the binding to a soluble form of the MET ectodomain in a solid phase binding assay (Gherardi, E. et al., Proc Natl Acad Sci USA 100, 12039-44 (2003)); the one on the right shows the specific activity of the mutant protein in cell migration assays. The results show that: (i) mutations affecting intra-protomeric contacts between the N and K1 domains (mutants NA, K1A and K1L) have a detectable but modest effect on receptor binding and biological activity, (ii) of the five mutants involved in receptor binding (Ne, NF, K1J, K1M, and K1N) only mutants carrying alanine substitutions at positions E195, R197 and Y198 in the K1 domain were completely inactive under the experimental conditions used (FIG. 8D); (iii) a number of mutants at the dimer interface exhibited greatly reduced activity, especially those carrying multiple substitutions in the linker (mutants LG and LH) or in the N or K1 domains (mutants NC, ND, K1G and K1H) or their combinations (mutants CA, CF, CG, and CH). A notable exception was the single linker mutation D 123A that was sufficient, alone, in reducing HGF/SF activity by more than 80%. Unexpectedly, most HGF/SF variants carrying the D 123A or complex mutations at the NK1 dimer interface also exhibited reduced receptor binding (FIG. 11D), a result confirmed with purified proteins produced from stable transfectants of selected mutants (FIG. 12).

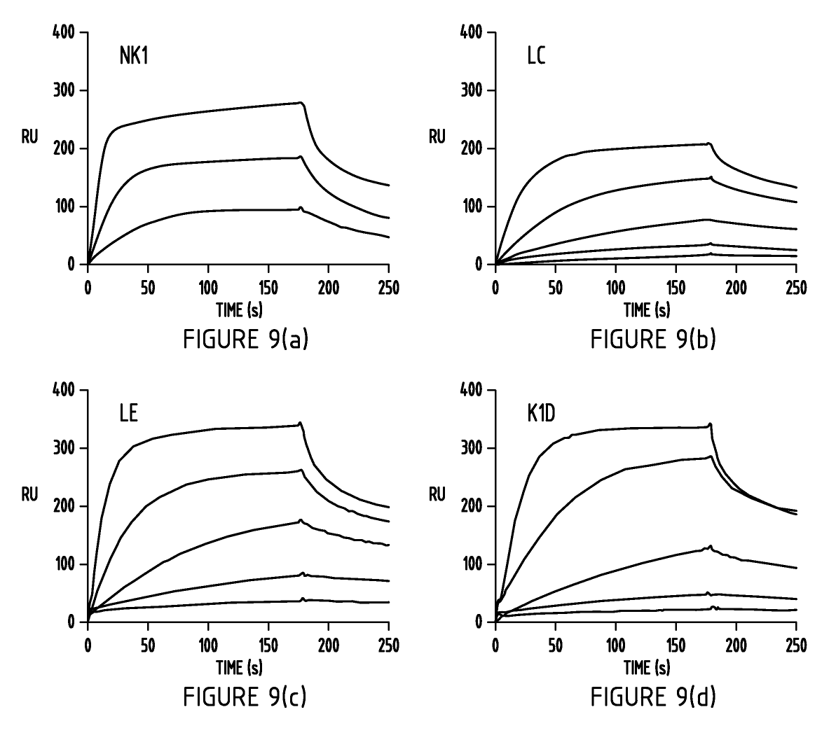

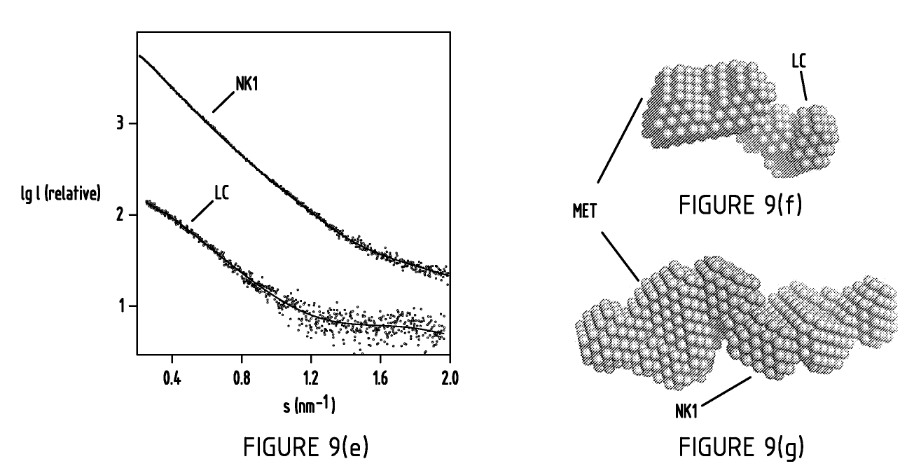

NK1 versions of the above mutants were expressed in the yeast P. pastoris and purified to homogeneity. The single linker mutants LC (Y 124A) and LE (N127A) bound MET with affinities indistinguishable from wild type NK1 in surface plasmon resonance experiments (1.83×10-7M for NK1, 1.49×10-7 M for the LC mutant and 0.83×10-7 M for the LE mutant) (FIGS. 9A-C). In marked contrast, the double mutants LF (D123A:Y124A) and LG (D123A:N127A) displayed >100 fold reduced MET binding affinity (data not shown). The binding affinity of the K1 mutant K1D (V140A:I142A) was comparable to wild type NK 1 (1.60×10-7 M). These results confirm those obtained with the full length constructs and demonstrate that mutations at the NK 1 binding interface have markedly different effects on MET binding.

The stoichiometry of complexes formed by NK 1 mutants at the dimer interface with MET567, a fragment of MET ectodomain carrying the ligand-binding-propeller domain (Gherardi, E. et al., 100, 12039-44 (2003)), was investigated by small angle X-ray scattering (SAXS). FIG. 9E shows experimental SAXS scattering profiles of wild type NK1 and the LC mutant in complex with MET567. The overall structural parameters computed from the SAXS are given in Table 3 and indicate that, in the presence of 12mer heparin, the complex formed by NK1 and MET567 is dimeric and has a 2:2 stoichiometry in solution whereas that formed by the linker mutant LC and MET567 has 1:1 stoichiometry. This finding is further supported by shape determination. Low resolution bead models of the wild type and LC complexes, reconstructed with MONSA (Svergun, D, Biophys J. 76, 2879-86 (1999)) neatly fit the experimental data (FIG. 9E and Table 4). Typical ab initio models reconstructed in independent runs of MONSA are shown in FIG. 9F (LC-MET567) and FIG. 9G (NK1-MET567). The latter model accommodates two copies of a 1:1 NK1-MET567 complex with the dimer built around the NK1 dimerisation interface and two molecules of MET567 located at the periphery.

TABLE 4

| | Rg, nm | MM, kDa | ^{D}max, nm | |
|---|---|---|---|---|
| LC + MET | 3.580.05 | 9010 | 12 | 1.58 |
| NK1 + MET | 5.260.05 | 21020 | 20 | 1.39 |

Rg, MM and Dmax are, respectively, radius of gyration, molecular mass and maximum size calculated from the scattering data. χ is the overall discrepancy between the experimental data and computed curves from the ab initio models.

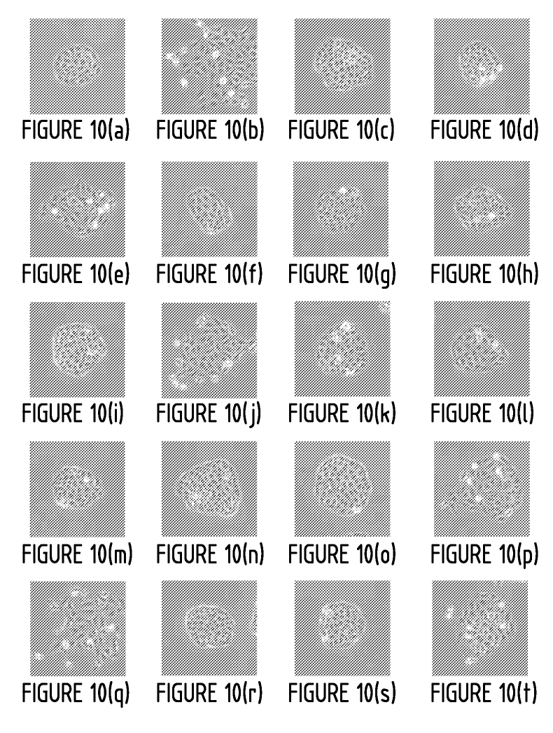

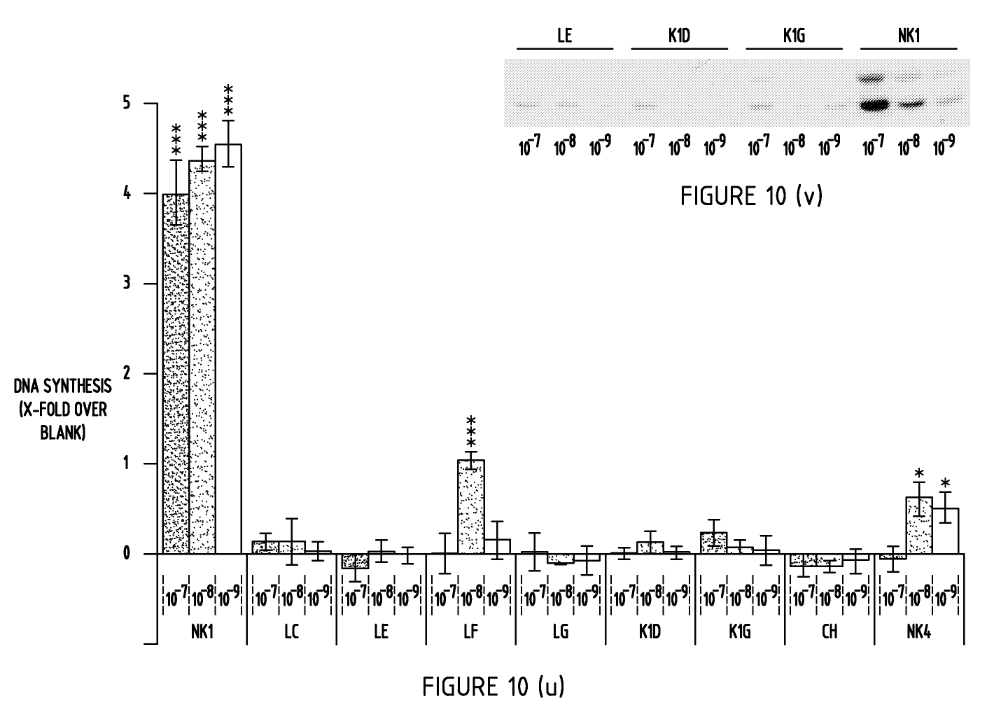

The ability of the NK 1 mutants to induce migration of MDCK cells or Erk1/2 phosphorylation and DNA synthesis in MK cells was studied next. The LC mutant displayed limited activity on MDCK cells at the highest concentration tested (FIG. 10C-10E), the LE mutant had no activity (FIG. 10D-10H). The LF mutant (D123A:Y124A), in spite of reduced MET binding, consistently produced a bell-shaped response both in cell migration (FIG. 10I-10K) and DNA synthesis assays (FIG. 10U). As expected from the results with the full length construct (FIG. 8D), the complex CH mutant was inactive (FIGS. 10L-N and 10U). The NK 1 versions of the receptor-binding mutants K1M (E195A:R197A:Y198A) and K1N (E159A:S161A:E195A:R197A:Y198A), however, displayed agonistic activity at concentrations of 10⁻⁸ M or higher (K1M) and of 10⁻⁷ M (K1N). These proteins, therefore were 100 to 1000 fold less active than wild type NK1 but the residual agonistic activity was readily measurable.

Finally, the NK1 mutants were assayed for receptor antagonistic activity. FIG. 11 shows that the linker LE mutant (N127A) at a concentration of 10⁻⁷ M could block completely the activity of HGF/SF on MDCK cells (FIG. 11H). As expected, both wild-type NK1 (FIG. 11C-D) and the K1N mutant (FIG. 11E-F) had no antagonistic activity. The antagonistic activity of the LE mutant and several other mutants at the dimer interface in DNA synthesis assays and Erk1/2 phosphorylation assays is shown in FIGS. 11I and 11J, respectively. The results demonstrated that both the linker mutants LC(Y124A), LE (N127A) and LG (D123A:1N127A) as well as the K1 mutants K1D (V140A:I142A) and K1G (K137:T139A:S 141:I142) effectively antagonize HGF/SF (FIG. 11I). Erk1/2 phosphorylation assays confirmed the antagonistic activity of the LE and K1D mutants and a partial activity for the LC mutant. In these assays the LG and K1G mutants were inactive suggesting that the binding kinetics and/or affinities of these mutants fail to block short-term activation of the MET receptor. Thus, here disclosed is the conversion of the NK1 receptor binding fragment of HGF/SF, a partial agonist, into a receptor antagonist.

Point mutations were introduced at the crystallographic dimer interface following the hypothesis that such mutations would disrupt dimerization, and hence activity, without affecting receptor binding. The single linker mutations Y124A and N127A or the double mutation V140A:I142A in the K1 domain had the phenotype hypothesized. The single linker mutation D123A and complex mutations at the dimer interface, however, also affected receptor binding. This result was unexpected and is discussed first.

The evidence that opposite faces of the NK1 protomer are responsible for receptor binding and dimer formation has a strong foundation in crystallographic (Chirgadze, D. Y. Nat Struct Biol, 6, 72-9 (1999); Lietha, D., Embo J., 20, 5543-55 (2001); Ultsch, M., Structure, 6, 1383-93 (1998); Watanabe, K, J. Mol. Bio, 319, 283-88 (2002) and mutagenesis studies (Hartmann, G [published erratum appears in Curr Biol 1998 Oct. 8; 8(20):R739], Curr Biol, 8, 125-34 (1998); Lokker, N. A., Protein Eng, 7, 895-903 (1994)). Without being bound by any particular theory, it is believed that the interdomain linker is flexible (Watanabe, K, J. Mol. Bio, 319, 283-88 (2002); Gherardi, E. et al. Proc Natl Acad Sci USA 103, 4046-51 (2006)) and the first 3 amino acids (K 122, D123, Y 124) form a 3/10 helix. Conceivably, the linker mutation D123A may disrupt the 3/10 helix and thus the rotation and orientation of the N and K1 domains affecting indirectly, receptor binding.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

Those skilled in the art will recognize, or be able to ascertain using no more than routine experimentation, many equivalents to the specific embodiments of the invention described herein. Such equivalents are intended to be encompassed by the following claims.

### REFERENCES

Birchmeier, C., Birchmeier, W., Gherardi, E., and Vande Woude, G. F. (2003). Met, metastasis, motility and more. Nat Rev Mol Cell Biol 4, 915-925.
Bladt, F., Riethmacher, D., Isenmann, S., Aguzzi, A., and Birchmeier, C. (1995). Essential role for the c-met receptor in the migration of myogenic precursor cells into the limb bud. Nature 376, 768-771.
Blume-Jensen, P., and Hunter, T. (2001). Oncogenic kinase signalling. Nature 411, 355-365.
Borowiak, M., Garratt, A. N., Wustefeld, T., Strehle, M., Trautwein, C., and Birchmeier, C. (2004). Met provides essential signals for liver regeneration. Proc Natl Acad Sci USA 101, 10608-10613.
Bottaro, D. P., Rubin, J. S., Faletto, D. L., Chan, A. M., Kmiecik, T. E., Vande Woude, G. F., and Aaronson, S. A. (1991). Identification of the hepatocyte growth factor receptor as the c-met proto-oncogene product. Science 251, 802-804.
Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., et al. (1998). Crystallography & NMR system: A new software suite for macromolecular structure determination. Acta Crystallogr D Biol Crystallogr 54, 905-921.
Cao, B., Su, Y., Oskarsson, M., Zhao, P., Kort, E. J., Fisher, R. J., Wang, L. M., and Vande Woude, G. F. (2001). Neutralizing monoclonal antibodies to hepatocyte growth factor/scatter factor (HGF/SF) display antitumor activity in animal models. Proc Natl Acad Sci U S A 98, 7443-7448.
Catlow, K., Deakin, J. A., Delehedde, M., Fernig, D. G., Gallagher, J. T., Pavao, M. S., and Lyon, M. (2003). Hepatocyte growth factor/scatter factor and its interaction with heparan sulphate and dermatan sulphate. Biochem Soc Trans 31, 352-353.
Chirgadze, D. Y., Hepple, J., Byrd, R. A., Sowdhamini, R., Blundell, T. L., and Gherardi, E. (1998). Insights into the structure of hepatocyte growth factor/scatter factor (HGF/SF) and implications for receptor activation. FEBS Lett 430, 126-129.
Cho, H. S., and Leahy, D. J. (2002). Structure of the extracellular region of HER3 reveals an interdomain tether. Science 297, 1330-1333.
Christensen, J. G., Schreck, R., Burrows, J., Kuruganti, P., Chan, E., Le, P., Chen, J., Wang, X., Ruslim, L., Blake, R., et al. (2003). A selective small molecule inhibitor of c-Met kinase inhibits c-Met-dependent phenotypes in vitro and exhibits cytoreductive antitumor activity in vivo. Cancer Res 63, 7345-7355.
Cioce, V., Csaky, K. G., Chan, A. M., Bottaro, D. P., Taylor, W. G., Jensen, R., Aaronson, S. A., and Rubin, J. S. (1996). Hepatocyte growth factor (HGF)/NK1 is a naturally occurring HGF/scatter factor variant with partial agonist/antagonist activity. J Biol Chem 271, 13110-13115.
Cooper, C. S., Park, M., Blair, D. G., Tainsky, M. A., Huebner, K., Croce, C. M., and Vande Woude, G. F. (1984). Molecular cloning of a new transforming gene from a chemically transformed human cell line. Nature 311, 29-33.
Duffy, S., Tsao, K. L., and Waugh, D. S. (1998). Site-specific, enzymatic biotinylation of recombinant proteins in Spodoptera frugiperda cells using biotin acceptor peptides. Anal Biochem 262, 122-128.
Ferguson, K. M., Berger, M. B., Mendrola, J. M., Cho, H. S., Leahy, D. J., and Lemmon, M. A. (2003). EGF activates its receptor by removing interactions that autoinhibit ectodomain dimerization. Mol Cell 11, 507-517.
Gao, C. F., Xie, Q., Su, Y. L., Koeman, J., Khoo, S. K., Gustafson, M., Knudsen, B. S., Hay, R., Shinomiya, N., and Vande Woude, G. F. (2005). Proliferation and invasion: plasticity in tumor cells. Proc Natl Acad Sci USA 102, 10528-10533.
Garrett, T. P., McKern, N. M., Lou, M., Elleman, T. C., Adams, T. E., Lovrecz, G. O., Zhu, H. J., Walker, F., Frenkel, M. J., Hoyne, P. A., et al. (2002). Crystal structure of a truncated epidermal growth factor receptor extracellular domain bound to transforming growth factor alpha. Cell 110, 763-773.
Gherardi, E., Gray, J., Stoker, M., Perryman, M., and Furlong, R. (1989). Purification of scatter factor, a fibroblast-derived basic protein that modulates epithelial interactions and movement. Proc Natl Acad Sci USA 86, 5844-5848.
Gherardi, E., Sandin, S., Petoukhov, M. V., Finch, J., Youles, M. E., Ofverstedt, L. G., Miguel, R. N., Blundell, T. L., Vande Woude, G. F., Skoglund, U., and Svergun, D. I. (2006). Structural basis of hepatocyte growth factor/scatter factor and MET signalling. Proc Natl Acad Sci USA 103, 4046-4051.
Gherardi, E., Youles, M. E., Miguel, R. N., Blundell, T. L., Tamele, L., Gough, J., Bandyopadhyay, A., Hartmann, G., and Butler, P. J. (2003). Functional map and domain structure of MET, the product of the c-met protooncogene and receptor for hepatocyte growth factor/scatter factor. Proc Natl Acad Sci USA 100, 12039-12044.
Holmes, O., Pillozzi, S., Deakin, J. A., Carafoli, F., Kemp, L., Butler, P. J., Lyon, M., and Gherardi, E. (2007). Insights into the structure/function of hepatocyte growth factor/scatter factor from studies with individual domains. J Mol Biol 367, 395-408.
Huh, C. G., Factor, V. M., Sanchez, A., Uchida, K., Conner, E. A., and Thorgeirsson, S. S. (2004). Hepatocyte growth factor/c-met signaling pathway is required for efficient liver regeneration and repair. Proc Natl Acad Sci USA 101, 4477-4482.
Jakubczak, J. L., LaRochelle, W. J., and Merlino, G. (1998). NK1, a natural splice variant of hepatocyte growth factor/scatter factor, is a partial agonist in vivo. Mol Cell Biol 18, 1275-1283.
Jones, T. A., Zou, J. Y., Cowan, S. W., and Kjeldgaard, M. (1991). Improved methods for building protein models in electron density maps and the location of errors in these models. Acta Crystallogr A 47 (Pt 2), 110-119.
Komada, M., Hatsuzawa, K., Shibamoto, S., Ito, F., Nakayama, K., and Kitamura, N. (1993). Proteolytic processing of the hepatocyte growth factor/scatter factor receptor by furin. FEBS Lett 328, 25-29.
Kong-Beltran, M., Stamos, J., and Wickramasinghe, D. (2004). The Sema domain of Met is necessary for receptor dimerization and activation. Cancer Cell 6, 75-84.
Lietha, D., Chirgadze, D. Y., Mulloy, B., Blundell, T. L., and Gherardi, E. (2001). Crystal structures of NK1-heparin complexes reveal the basis for NK1 activity and enable engineering of potent agonists of the MET receptor. Embo J 20, 5543-5555.
Lokker, N. A., and Godowski, P. J. (1993). Generation and characterization of a competitive antagonist of human hepatocyte growth factor, HGF/NK1. J Biol Chem 268, 17145-17150.
Lokker, N. A., Presta, L. G., and Godowski, P. J. (1994). Mutational analysis and molecular modeling of the N-terminal kringle-containing domain of hepatocyte growth factor identifies amino acid side chains important for interaction with the c-Met receptor. Protein Eng 7, 895-903.
Lyon, M., Deakin, J. A., Lietha, D., Gherardi, E., and Gallagher, J. T. (2004). The interactions of hepatocyte growth factor/scatter factor and its NK1 and NK2 variants with glycosaminoglycans using a modified gel mobility shift assay. Elucidation of the minimal size of binding and activatory oligosaccharides. J Biol Chem 279, 43560-43567.
Mars, W. M., Zarnegar, R., and Michalopoulos, G. K. (1993). Activation of hepatocyte growth factor by the plasminogen activators uPA and tPA. Am J Pathol 143, 949-958.
Matsumoto, K., and Nakamura, T. (2003). NK4 (HGF-antagonist/angiogenesis inhibitor) in cancer biology and therapeutics. Cancer Sci 94, 321-327.
Mazzone, M., Basilico, C., Cavassa, S., Pennacchietti, S., Risio, M., Naldini, L., Comoglio, P. M., and Michieli, P. (2004). An uncleavable form of pro-scatter factor suppresses tumor growth and dissemination in mice. J Clin Invest 114, 1418-1432.
Michieli, P., Mazzone, M., Basilico, C., Cavassa, S., Sottile, A., Naldini, L., and Comoglio, P. M. (2004). Targeting the tumor and its microenvironment by a dual-function decoy Met receptor. Cancer Cell 6, 61-73.
Montesano, R., Soriano, J. V., Malinda, K. M., Ponce, M. L., Bafico, A., Kleinman, H. K., Bottaro, D. P., and Aaronson, S. A. (1998). Differential effects of hepatocyte growth factor isoforms on epithelial and endothelial tubulogenesis. Cell Growth Differ 9, 355-365.
Nakamura, T., Nishizawa, T., Hagiya, M., Seki, T., Shimonishi, M., Sugimura, A., Tashiro, K., and Shimizu, S. (1989). Molecular cloning and expression of human hepatocyte growth factor. Nature 342, 440-443.
Ogiso, H., Ishitani, R., Nureki, O., Fukai, S., Yamanaka, M., Kim, J. H., Saito, K., Sakamoto, A., Inoue, M., Shirouzu, M., and Yokoyama, S. (2002). Crystal structure of the complex of human epidermal growth factor and receptor extracellular domains. Cell 110, 775-787.
Otwinowski, Z., and Minor, W. (1997). Processing of x-ray diffraction data collected in oscillation mode. Methods in Enzymology 276, 307-326.
Park, M., Dean, M., Cooper, C. S., Schmidt, M., O'Brien, S. J., Blair, D. G., and Vande Woude, G. F. (1986). Mechanism of met oncogene activation. Cell 45, 895-904.
Pediaditakis, P., Monga, S. P., Mars, W. M., and Michalopoulos, G. K. (2002). Differential mitogenic effects of single chain hepatocyte growth factor (HGF)/scatter factor and HGF/NK1 following cleavage by factor Xa. J Biol Chem 277, 14109-14115.
Pellegrini, L., Burke, D. F., von Delft, F., Mulloy, B., and Blundell, T. L. (2000). Crystal structure of fibroblast growth factor receptor ectodomain bound to ligand and heparin. Nature 407, 1029-1034.
Petrelli, A., Circosta, P., Granziero, L., Mazzone, M., Pisacane, A., Fenoglio, S., Comoglio, P. M., and Giordano, S. (2006). Ab-induced ectodomain shedding mediates hepatocyte growth factor receptor down-regulation and hampers biological activity. Proc Natl Acad Sci USA 103, 5090-5095.
Rubin, J. S., Day, R. M., Breckenridge, D., Atabey, N., Taylor, W. G., Stahl, S. J., Wingfield, P. T., Kaufman, J. D., Schwall, R., and Bottaro, D. P. (2001). Dissociation of heparan sulfate and receptor binding domains of hepatocyte growth factor reveals that heparan sulfate-c-met interaction facilitates signaling. J Biol Chem 276, 32977-32983.
Schlessinger, J. (2002). Ligand-induced, receptor-mediated dimerization and activation of EGF receptor. Cell 110, 669-672.
Schlessinger, J., Plotnikov, A. N., Ibrahimi, O. A., Eliseenkova, A. V., Yeh, B. K., Yayon, A., Linhardt, R. J., and Mohammadi, M. (2000). Crystal structure of a ternary FGF-FGFR-heparin complex reveals a dual role for heparin in FGFR binding and dimerization. Mol Cell 6, 743-750.
Schmidt, C., Bladt, F., Goedecke, S., Brinkmann, V., Zschiesche, W., Sharpe, M., Gherardi, E., and Birchmeier, C. (1995). Scatter factor/hepatocyte growth factor is essential for liver development. Nature 373, 699-702.
Schwall, R. H., Chang, L. Y., Godowski, P. J., Kahn, D. W., Hillan, K. J., Bauer, K. D., and Zioncheck, T. F. (1996). Heparin induces dimerization and confers proliferative activity onto the hepatocyte growth factor antagonists NK1 and NK2. J Cell Biol 133, 709-718.
Stamos, J., Lazarus, R. A., Yao, X., Kirchhofer, D., and Wiesmann, C. (2004). Crystal structure of the HGF beta-chain in complex with the Sema domain of the Met receptor. Embo J 23, 2325-2335.
Ultsch, M., Lokker, N. A., Godowski, P. J., and de Vos, A. M. (1998). Crystal structure of the NK1 fragment of human hepatocyte growth factor at 2.0 A resolution. Structure 6, 1383-1393.
Weiss, A., and Schlessinger, J. (1998). Switching signals on or off by receptor dimerization. Cell 94, 277-280.
Wiesmann, C., Fuh, G., Christinger, H. W., Eigenbrot, C., Wells, J. A., and de Vos, A. M. (1997). Crystal structure at 1.7 A resolution of VEGF in complex with domain 2 of the Flt-1 receptor. Cell 91, 695-704.
Wiesmann, C., Ultsch, M. H., Bass, S. H., and de Vos, A. M. (1999). Crystal structure of nerve growth factor in complex with the ligand-binding domain of the TrkA receptor. Nature 401, 184-188.
Xie, Q., Gao, C. F., Shinomiya, N., Sausville, E., Hay, R., Gustafson, M., Shen, Y., Wenkert, D., and Vande Woude, G. F. (2005). Geldanamycins exquisitely inhibit HGF/SF-mediated tumor cell invasion. Oncogene 24, 3697-3707.
Zhang, Y. W., Su, Y., Volpert, O. V., and Vande Woude, G. F. (2003). Hepatocyte growth factor/scatter factor mediates angiogenesis through positive VEGF and negative thrombospondin 1 regulation. Proc Natl Acad Sci USA 100, 12718-12723.
Zhou, H., Casas-Finet, J. R., Heath Coats, R., Kaufman, J. D., Stahl, S. J., Wingfield, P. T., Rubin, J. S., Bottaro, D. P., and Byrd, R. A. (1999). Identification and dynamics of a heparin-binding site in hepatocyte growth factor. Biochemistry 38, 14793-14802.

What is claimed is:

1. An NK1-based polypeptide comprising the amino acids of any of SEQ ID NO: 8-53, 58, or 59 or SEQ ID NO: 8-53, 58, or 59 with one or more conservative amino acid substitutions, wherein the polypeptide modulates Met activity as compared to wild type NK1; and derivatives thereof.

2. The NK1-based polypeptide of claim 1 comprising the amino acids of any of SEQ ID NO: 8-13, 20-36, 58, and 59 wherein the polypeptide is a Met antagonist; and derivatives thereof.

3. An NK1-based polypeptide comprising amino acids of any of SEQ ID NO: 54 or 55, or SEQ ID NO: 54 or 55 with one or more conservative amino acid substitutions; and derivatives thereof.

4. The NK1-based polypeptide of claim 3 comprising amino acids of SEQ ID NO: 54 or SEQ ID NO: 54 with one or more conservative amino acid substitutions; and derivatives thereof.

5. An NK1-based polypeptide comprising amino acids of SEQ ID NO: 1 wherein the amino acid at one of wild type NK1 amino acid positions 77, 82, 83, 85, 122, 123, 124, 126, 127, 134, 137, 139, 140, 141, and 142 is substituted with an amino acid not normally found at that position in SEQ ID NO: 1, and wherein the polypeptide decreases Met activity as compared to wild type NK1; and derivatives thereof.

6. An NK1-based polypeptide comprising amino acids of SEQ ID NO: 1 wherein the amino acid at two of wild type NK1 amino acid positions 77, 82, 83, 85, 122, 123, 124, 126, 134, 137, 139, 141 and 142 is substituted with an amino acid not normally found at that position in SEQ ID NO: 1, and wherein the polypeptide decreases Met activity as compared to wild type NK1; and derivatives thereof.

7. An NK1-based polypeptide comprising amino acids of SEQ ID NO: 1 wherein the amino acid at three of wild type NK1 amino acid positions 77, 83, 85, 122, 123, 124, 126, 127, 134, 137, 139, 140, 141, and 142 is substituted with an amino acid not normally found at that position in SEQ ID NO: 1, and wherein the polypeptide decreases Met activity as compared to wild type NK1; and derivatives thereof.

8. A composition comprising the polypeptide of any of claims 1-7 and a pharmaceutically acceptable diluent or carrier.

9. A method for reducing Met activity in a cell comprising introducing the polypeptide of any of claims 1, 2, 5, 6, or 7 to the cell.

10. The method of claim 9 wherein the cell is a tumor cell.

11. A method for increasing Met activity in a cell comprising introducing the polypeptide of claims 3 or 4 to the cell.

12. A method for designing an antagonist by converting a growth factor from a receptor tyrosine kinase agonist to an antagonist, wherein the native receptor dimerizes or oligomerizes to become active, comprising the steps:
identifying a region of said growth factor that promotes said dimerization or oligomerization of said receptor;
providing growth factor mutants in which at least one amino acid in said region of said growth factor that promotes said dimerization or oligomerization has been substituted with another amino acid;
screening said mutants to determine receptor dimerization or oligomerization activity and to determine the binding affinity of the mutants to the receptor, and selecting one or more mutants that have a decreased ability as compared to wild type growth factor to promote dimerization or oligomerization of the receptor but retain the ability to bind the receptor.

13. The method of claim 12 wherein the receptor tyrosine kinase is Met.

14. The method of claim 12 wherein the receptor tyrosine kinase is selected from the group consisting of epidermal growth factor receptor (EGFR), nerve growth factor receptor (NGFR), vascular endothelial growth factor receptor (VEGFR), fibroblast growth factor receptor (FGFR), stem cell factor receptor, and macrophage-stimulating protein receptor (RON).

15. The method of claims 12 wherein a proximity assay is used to determine receptor dimerization or oligomerization activity or to determine the binding affinity of said mutants to the receptor.

16. A polynucleotide coding for the polypeptide or derivative thereof, of any one of claims 1-7.

17. An expression vector comprising the polynucleotide, or derivative thereof, of claim 16 operably linked to a promoter.

18. A host cell carrying the vector of claim 17.

19. A method of treating a patient in need of control of cell growth, cell proliferation, cell survival, or cell motility, said method comprising administering to said patient a therapeutically effective amount of the polypeptide or derivative of any one of claims 1-7.

20. A method of treating a patient in need of anti-cancer therapy, said method comprising administering to said patient a therapeutically effective amount of the polypeptide or derivative of any one of claims 1, 2, 5, 6, or 7.

21. A kit for providing a therapeutic dose of bioactive agent for use in treating a patient, comprising:
a container; and
a volume of material stored within the container in substantially sterile form; wherein the material comprises the polypeptide of any one of claims 1-7 or a derivative thereof.

22. A method for treating an abnormal cellular proliferation condition associated with a region of tissue in a living patient, comprising:
delivering a therapeutically effective amount of a material to the region of tissue;
inhibiting the abnormal cellular proliferation condition with the therapeutic amount of the material;
wherein the material comprises the polypeptide or derivative of any one of claims 1, 2, 5, 6, or 7, or a derivative thereof.

23. The method of claim 22, wherein the region of tissue comprises a tumor, and the abnormal cellular proliferation condition comprises cancer, and further wherein; the therapeutic amount of the material is delivered to the tumor; and proliferation of the cancer is inhibited with the therapeutic dose of the material.
